SEQUENCE LISTING (50 sequences)

SEQ ID NO: 1 - protein, 17 aa, Artificial Sequence
  Description of Artificial Sequence Peptide isolated from colostrinin
LQTPQPLLQVMMEPQGD

SEQ ID NO: 2 - protein, 11 aa, Artificial Sequence
  Description of Artificial Sequence Peptide isolated from colostrinin
MPQNFYKLPQM

SEQ ID NO: 3 - protein, 15 aa, Artificial Sequence
  Description of Artificial Sequence Peptide isolated from colostrinin
VLEMKFPPPPQETVT

SEQ ID NO: 4 - protein, 15 aa, Artificial Sequence
  Description of Artificial Sequence Peptide isolated from colostrinin
LKPFPKLKVEVFPFP

SEQ ID NO: 5 - protein, 4 aa, Artificial Sequence
  Description of Artificial Sequence Peptide isolated from colostrinin
SEQP

SEQ ID NO: 6 - protein, 3 aa, Artificial Sequence
  Description of Artificial Sequence Peptide isolated from colostrinin
DKE

SEQ ID NO: 7 - protein, 7 aa, Artificial Sequence
  Description of Artificial Sequence Peptide isolated from colostrinin
DPPPPQS

SEQ ID NO: 8 - protein, 3 aa, Artificial Sequence
  Description of Artificial Sequence Peptide isolated from colostrinin
LNF

SEQ ID NO: 9 - protein, 7 aa, Artificial Sequence
  Description of Artificial Sequence Peptide isolated from colostrinin
VLPPNVG

SEQ ID NO: 10 - protein, 7 aa, Artificial Sequence
  Description of Artificial Sequence Peptide isolated from colostrinin
KYKLQPE

SEQ ID NO: 11 - protein, 5 aa, Artificial Sequence
  Description of Artificial Sequence Peptide isolated from colostrinin
SEEMP

SEQ ID NO: 12 - protein, 6 aa, Artificial Sequence
  Description of Artificial Sequence Peptide isolated from colostrinin
DSQPPV

SEQ ID NO: 13 - protein, 5 aa, Artificial Sequence
  Description of Artificial Sequence Peptide isolated from colostrinin
FPPPK

SEQ ID NO: 14 - protein, 5 aa, Artificial Sequence
  Description of Artificial Sequence Peptide isolated from colostrinin
VVMEV

SEQ ID NO: 15 - protein, 15 aa, Artificial Sequence
  Description of Artificial Sequence Peptide isolated from colostrinin
DLEMPVLPVEPFPFV

SEQ ID NO: 16 - protein, 12 aa, Artificial Sequence
  Description of Artificial Sequence Peptide isolated from colostrinin
LFFFLPVVNVLP

SEQ ID NO: 17 - protein, 7 aa, Artificial Sequence
  Description of Artificial Sequence Peptide isolated from colostrinin
MQPPPLP

SEQ ID NO: 18 - protein, 18 aa, Artificial Sequence
  Description of Artificial Sequence Peptide isolated from colostrinin
DQPPDVEKPDLQPFQVQS

SEQ ID NO: 19 - protein, 10 aa, Artificial Sequence
  Description of Artificial Sequence Peptide isolated from colostrinin
VYPFTGPIPN

SEQ ID NO: 20 - protein, 9 aa, Artificial Sequence
  Description of Artificial Sequence Peptide isolated from colostrinin
SLPQNILPL

SEQ ID NO: 21 - protein, 10 aa, Artificial Sequence
  Description of Artificial Sequence Peptide isolated from colostrinin
TQTPVVVPPF

SEQ ID NO: 22 - protein, 18 aa, Artificial Sequence
  Description of Artificial Sequence Peptide isolated from colostrinin
LQPEIMGVPKVKETMVPK

SEQ ID NO: 23 - protein, 18 aa, Artificial Sequence
  Description of Artificial Sequence Peptide isolated from colostrinin
HKEMPFPKYPVEPFTESQ

SEQ ID NO: 24 - protein, 18 aa, Artificial Sequence
  Description of Artificial Sequence Peptide isolated from colostrinin
SLTLTDVEKLHLPLPLVQ

SEQ ID NO: 25 - protein, 7 aa, Artificial Sequence
  Description of Artificial Sequence Peptide isolated from colostrinin
SWMHQPP

SEQ ID NO: 26 - protein, 10 aa, Artificial Sequence
  Description of Artificial Sequence Peptide isolated from colostrinin
QPLPPTVMFP

SEQ ID NO: 27 - protein, 15 aa, Artificial Sequence
  Description of Artificial Sequence Peptide isolated from colostrinin
MHQPPQPLPPTVMFP

SEQ ID NO: 28 - protein, 6 aa, Artificial Sequence
  Description of Artificial Sequence Peptide isolated from colostrinin
PQSVLS

SEQ ID NO: 29 - protein, 22 aa, Artificial Sequence
  Description of Artificial Sequence Peptide isolated from colostrinin
LSQPKVLPVPQKAVPQRDMPIQ

SEQ ID NO: 30 - protein, 7 aa, Artificial Sequence
  Description of Artificial Sequence Peptide isolated from colostrinin
AFLLYQE

SEQ ID NO: 31 - protein, 13 aa, Artificial Sequence
  Description of Artificial Sequence Peptide isolated from colostrinin
FLLYQEPVLGPVR

SEQ ID NO: 32 - protein, 8 aa, Artificial Sequence
  Description of Artificial Sequence Peptide isolated from colostrinin
RGPFPILV

SEQ ID NO: 33 - protein, 18 aa, Artificial Sequence
  Description of Artificial Sequence Peptide isolated from colostrinin
ATFNRYQDDHGEEILKSL

SEQ ID NO: 34 - protein, 18 aa, Artificial Sequence
  Description of Artificial Sequence Peptide isolated from colostrinin
CLQTPQPLLQVMMEPQGD

SEQ ID NO: 35 - protein, 12 aa, Artificial Sequence
  Description of Artificial Sequence Peptide isolated from colostrinin
CMPQNFYKLPQM

SEQ ID NO: 36 - protein, 16 aa, Artificial Sequence
  Description of Artificial Sequence Peptide isolated from colostrinin
CVLEMKFPPPPQETVT

SEQ ID NO: 37 - protein, 16 aa, Artificial Sequence
  Description of Artificial Sequence Peptide isolated from colostrinin
CLKPFPKLKVEVFPFP

SEQ ID NO: 38 - protein, 8 aa, Artificial Sequence
  Description of Artificial Sequence Peptide isolated from colostrinin
SEQPGGGC

SEQ ID NO: 39 - protein, 9 aa, Artificial Sequence
  Description of Artificial Sequence Peptide isolated from colostrinin
CGVLPPNVG

SEQ ID NO: 40 - protein, 10 aa, Artificial Sequence
  Description of Artificial Sequence Peptide isolated from colostrinin
CGGGKYKLQE

SEQ ID NO: 41 - protein, 9 aa, Artificial Sequence
  Description of Artificial Sequence Peptide isolated from colostrinin
CGGGSEEMP

SEQ ID NO: 42 - protein, 10 aa, Artificial Sequence
  Description of Artificial Sequence Peptide isolated from colostrinin
CGGGDSQPPV

SEQ ID NO: 43 - protein, 10 aa, Artificial Sequence
  Description of Artificial Sequence Peptide isolated from colostrinin
CFPPPKGGGC

SEQ ID NO: 44 - protein, 9 aa, Artificial Sequence
  Description of Artificial Sequence Peptide isolated from colostrinin
CGGGVVMEV

SEQ ID NO: 45 - protein, 16 aa, Artificial Sequence
  Description of Artificial Sequence Peptide isolated from colostrinin
CDLEMPVLPVEPFPFV

SEQ ID NO: 46 - protein, 14 aa, Artificial Sequence
  Description of Artificial Sequence Peptide isolated from colostrinin
CLFFFLPVVNVLPI

SEQ ID NO: 47 - protein, 8 aa, Artificial Sequence
  Description of Artificial Sequence Peptide isolated from colostrinin
CMQPPPLP

SEQ ID NO: 48 - protein, 19 aa, Artificial Sequence
  Description of Artificial Sequence Peptide isolated from colostrinin
CDQPPDVEKPDLQPFQVQS

SEQ ID NO: 49 - protein, 9 aa, Artificial Sequence
  Description of Artificial Sequence Peptide isolated from colostrinin
CGAFLLYQE

SEQ ID NO: 50 - protein, 19 aa, Artificial Sequence
  Description of Artificial Sequence Peptide isolated from colostrinin
CATFNRYQDDHGEEILKSL

(19) United States
(12) Patent Application Publication
(10) Pub. No.: US 2006/0154871 A1
(43) Pub. Date: Jul. 13, 2006

(54) Peptides

(75) Inventor: Jerzy A. Georgiades, Houston, TX (US)

Correspondence Address: RANKIN, HILL, PORTER & CLARK, LLP, 925 EUCLID AVENUE, SUITE 700, CLEVELAND, OH, 44115-1405, US

(73) Assignee: ReGen Therapeutics PIc, London (GB)

(21) Appl. No.: 11/247,488

(22) Filed: Oct. 11, 2005

Related U.S. Application Data
(63) Continuation of application No. 09/980,347, filed on Oct. 22, 2002, now abandoned.

(30) Foreign Application Priority Data
Jun. 2, 1999 (GB) 9912852.2

(51) Int. Cl. A61K 38/10; A61K 38/08; C07K 7/08; C07K 7/06

(52) U.S. Cl. 514/13; 514/14; 530/326; 530/327; 530/328; 530/329; 514/15; 514/16; 514/17

(57) ABSTRACT

The amino acid sequence of several peptides present in Colostrinin is disclosed. These peptides are useful, inter alia, in the treatment of disorders of the immune system and the central nervous system.

The present application is a continuation of U.S. application Ser. No. 09/980,347, which was filed on Nov. 30, 2001 under 35 U.S.C. §371 (the 35 U.S.C. §371 requirements were met on Oct. 22, 2002) as a U.S. National Stage application of PCT/GB00/02128, which International Application was filed on Jun. 2, 2000, and claims priority to GB 9912852.2, which was filed Jun. 2, 1999.

The present application includes a sequence listing, which is hereby incorporated by reference. The sequence listing is written on paper and recorded on a compact disc accompanying the application as a file entitled “Peptides.txt”. The file was created on Sep. 29, 2005 at 9:10 a.m. and contains 15 KB of data. The file contents comply with the American Standard Code for Information Interchange (ASCII) and can be viewed using an IBM-PC compatible computer using the MS-Windows operating system. The sequence listing information recorded in computer readable form is identical to the sequence listing information written on paper.

### BACKGROUND OF THE INVENTION

1. Field of Invention

The present invention relates to peptides. More particularly the invention relates to certain peptides isolated from Colostrinin. The invention also relates to therapeutic uses of the peptides and to antibodies derived therefrom.

2. Description of Related Art

Colostrum is the thick, yellowish fluid produced by a mammalian mother's breasts during the first few days after childbirth. It is the first lacteal secretion post parturition and it contains a high concentration of immunoglobulins (IgG, IgM and IgA) and other proteins. It is replaced by mature breast milk about four to five days after birth. Compared with mature breast milk, colostrum contains low sugar and iron, but is rich in lipids, proteins, mineral salts, vitamins and immunoglobulins. Colostrum also contains various floating cells such as granular and stromal cells, neutrophils, monocyte/macrophages and lymphocytes and includes growth factors, hormones, cytokines and polypeptide complexes.

Various factors have been isolated and characterised from mammalian colostrum. In 1974, Janusz et al (FEES Lett., 49, 276-279) isolated a proline-rich polypeptide (PRP) from ovine colostrum. It has since been discovered that mammals other than sheep have analogues of PRP as a component of their colostrum. PRP has since been called Colostrinin (and is sometimes called Colostrinine).

M. Janusz & J. Lisowski in “Proline-Rich Polypeptide (PRP)— an Immunomodulatory Peptide from Ovine Colostrum” (Archivum Immunologiae et Therapiae Experimentalis, 1993, 41, 275-279) mentioned that PRP from ovine colostrum has immunotropic activity in mice.

A. Dubowska-Inglot et al in “Colostrinine: a praline-rich polypeptide from ovine colostrum is a modest cytokine inducer in human leukocytes” (Archivum Immunologiae et Therapiae Experimentalis, 1996, 44, 215-224) discussed the use of Colostrinin in the treatment of Alzheimer's disease. The use of Colostrinin in the treatment of Alzheimer's disease, and other conditions, was also discussed in WO-A-98/14473 and in “Colostrinin: a Proline-Rich Polypeptide (PRP) Complex isolated from Ovine Colostrum for Treatment of Alzheimer's Disease. A Double-Blind, Placebo-Controlled Study”, Leszek, J. et al, Archivum Immunologiae et Therapiae Experimental is, 1999, 47, 377-385.

Colostrinin, in its natural form, is obtained from mammalian colostrum. As described in WO-A-98/14473, analysis by electrophoresis and chromatography has shown that Colostrinin has the following properties:
(i) it has a molecular weight in the range 16,000 to 26,000 Daltons (this was shown by electrophoresis in the presence of SDS);
(ii) it is a dimer or trimer of sub-units each sub-unit having a molecular weight in the range 5,000 to 10,000 Daltons (this was shown by acrylamide gel electrophoresis in the presence of SDS);
   (iii) it contains proline, and the amount of proline is greater than the amount of any other single amino acid (this can be shown by conventional amino acid analysis).

By means of these techniques it was shown that ovine Colostrinin has a molecular weight of about 18,000 Daltons, is made up of three non-covalently linked sub-units each having a molecular weight of about 6,000 Daltons and includes about 22 wt % proline. The amino-acid composition of ovine Colostrinin was shown to be made up of the following number of residues per sub-unit: lysine-2, histidine-1, arginine-0, aspartic acid-2, threonine-4, serine-3, glutamic acid-6, proline-11, glycine-2, alanine-0, valine-5, methionine-2, isoleucine-2, leucine-6, tyrosine-1, phenylalanine-3 and cysteine-0.

### BRIEF SUMMARY OF THE INVENTION

We have now further analysed the composition of Colostrinin in order to try to identify its components, so that a synthetic form of Colostrinin can be produced.

We have concluded that Colostrinin contains peptide fragments from at least two different proteins: annexin; and β-casein. In addition, Colostrinin contains a number of other peptide fragments which do not have any known precursor protein; these amino acid sequences may be derived from an unknown precursor protein, or they may have no precursor protein. It is believed that some of the peptide sequences are from a β-casein homologue.

### BRIEF DESCRIPTION OF THE DRAWINGS

FIG. 1 is a Matrix-Assisted Laser Desorption Time-of-Flight Mass Spectroscopy (LDMS) spectra of NH₂-(Ac)CLQTPQPLLQVMMEPQGD-OH as synthesized in Example 3.

FIG. 2 is a LDMS spectra of NH₂-(Ac)CMPQNFYKLPQM-OH as synthesized in Example 3.

FIG. 3 is a LDMS spectra of NH₂-(Ac)CVLEMKFPPPPQETVT-OH as synthesized in Example 3.

FIG. 4 is a LDMS spectra of NH₂-(Ac)CLKPFPKLKVEVFPFP-OH as synthesized in Example 3.

FIG. 5 is a LDMS spectra of NH₂-SEQPGGGC-OH as synthesized in Example 3.

FIG. 6 is a LDMS spectra of NH₂-(Ac)CGVLPPNVG-OH as synthesized in Example 3.

FIG. 7 is a LDMS spectra of NH₂-(Ac)CGGGKYKLQE-OH as synthesized in Example 3.

FIG. 8 is a LDMS spectra of NH₂-(Ac)CGGGSEEMP(amide)-OH as synthesized in Example 3.

FIG. 9 is a LDMS spectra of NH₂-(Ac)CGGGDSQPPV-OH as synthesized in Example 3.

FIG. 10 is a LDMS spectra of NH₂-CFPPPKGGGC-OH as synthesized in Example 3.

FIG. 11 is a LDMS spectra of NH₂-(Ac)CGGGWMEV-OH as synthesized in Example 3.

FIG. 12 is a LDMS spectra of NH₂-(Ac)CDLEMPVLPVEPFPFV-OH as synthesized in Example 3.

FIG. 13 is a LDMS spectra of NH₂-(Ac)CLFFFLPWNVLPI-OH as synthesized in Example 3.

FIG. 14 is a LDMS spectra of NH₂-(Ac)CMQPPPLP-OH as synthesized in Example 3.

FIG. 15 is a LDMS spectra of NH₂-(Ac)CDQPPDVEKPDLQPFQVQS-OH as synthesized in Example 3.

FIG. 16 is a LDMS spectra of NH₂-(Ac)CGAFLLYQE-OH as synthesized in Example 3.

FIG. 17 is a LDMS spectra of NH₂-(Ac)CATFNRYQDDHGEEILKSL-OH as synthesized in Example 3.

FIG. 18 is a LDMS spectra of NH₂-DPPPQSGGGC-OH as synthesized in Example 3.

### DETAILED DESCRIPTION OF THE INVENTION

According to one aspect of the present invention there is provided a peptide having one of the following amino, acid sequences A-1 to D-1:

Group A: Peptides of unknown precursor

| | |
|---|---|
| A-1 | LQTPQPLLQVMMEPQGD |
| A-2 | MPQNFYKLPQM |
| A-3 | VLEMKFPPPPQETVT |
| A-4 | LKPFPKLKVEVFPFP |
| A-5 | SEQP |
| A-6 | DKE |
| A-7 | DPPPPQS |
| A-8 | LNF |

Group B: Peptides (possibly) having β-casein homologue precursor

| | |
|---|---|
| B-1 | VLPPNVG |
| B-2 | KYKLQPE |
| B-3 | SEEMP |
| B-4 | DSQPPV |
| B-5 | FPPPK |
| B-6 | VVMEV |
| B-7 | DLEMPVLPVEPFPFV |
| B-8 | LFFFLPVVNVLP |
| B-9 | MQPPPLP |
| B-10 | DQPPDVEKPDLQPFQVQS |

Group C: Peptides having β-casein precursor

| | | |
|---|---|---|
| C-1 | VYPFTGPIPN | (Casein Position 74-83) |
| C-2 | SLPQNILPL | (Casein Position 84-92) |
| C-3 | TQTPVVVPPF | (Casein Position 93-102) |
| C-4 | LQPEIMGVPKVKETMVPK | (Casein Position 103-120) |
| C-5 | HKEMPFPKYPVEPFTESQ | (Casein Position 121-138) |
| C-6 | SLTLTDVEKLHLPLPLVQ | (Casein Position 139-156) |
| C-7 | SWMHQPP | (Casein Position 157-163) |
| C-8 | QPLPPTVMFP | (Casein Position 164-173) |
| C-9 | MHQPPQPLPPTVMFP | (casein Position 159-173) |
| C-10 | PQSVLS | (Casein Position 174-179) |
| C-11 | LSQPKVLPVPQKAVPQRDMPIQ | (Casein Position 180-201) |
| C-12 | AFLLYQE | (Casein Position 202-208) |
| C-13 | FLLYQEPVLGPVR | (Casein Position 203-214) |
| C-14 | RGPFPILV | (Casein Position 214-222) |

Group D: Peptides having annexin precursor

| | | |
|---|---|---|
| D-1 | ATFNRYQDDHGEEILKSL | (Annexin Position 203-220) |

It is possible that the, peptides in group A are also derived from the β-casein homologue, but there is currently no evidence to support this conclusion.

These peptides may be provided in substantially isolated form. Furthermore, a composition may be provided which contains two or more of the above peptides, in combination.

In respect of the peptides A-1 to B-10, the invention further includes any peptide which includes the specified amino acid sequence. In respect of the peptides A1 to D1, the invention further comprises any peptide which includes an amino-terminal amino acid sequence corresponding to the specified sequence. Thus, with reference to peptide A-1, for example, the invention encompasses any peptide having the N-terminal amino acid sequence LQTPQPLLQVMMEPQGD; the same applies to peptides A-2 to D-1. For the avoidance of doubt, it is stated that the amino-terminal end is on the left hand side of the sequence, in accordance with the usual convention. It will be appreciated that any of the specified amino acid sequences may be provided with an inert amino acid sequence on the amino-terminal and/or the carboxy-terminal end thereof. The invention further includes physiologically acceptable active derivatives of the peptides.

The peptides can be obtained by a number of techniques. In one embodiment, they can be prepared naturally by isolation from Colostrinin or colostrum. In a preferred embodiment, they are prepared by a conventional technique for peptide synthesis, such as by solid-phase or liquid-phase peptide synthesis. Alternatively, the gene sequence encoding the peptides can be constructed by known techniques such as expression vectors or plasmids and transfected into suitable microorganisms that will express the DNA sequences, whereby the peptides can be later extracted from the medium in which the microorganisms are grown. Thus, the invention also embraces a DNA sequence encoding the peptides described above, and a recombinant vector prepared by inserting said DNA in a vector.

The peptides, either alone or in combination with one another, have a number of therapeutic uses.

In one advantageous embodiment, one or more of peptides A-1 to D-1 may be used in the treatment of disorders of the central nervous system, particularly chronic disorders of the central nervous system. The disorders of the central nervous system that may be treated include neurological disorders and mental disorders. Examples of neurological disorders that may, with advantage, be treated include dementia, and also disorders that cause dementia, such as neurodegenerative disorders. Neurodegenerative disorders include, for example, senile dementia and motor neurone disease; Parkinson's disease is an example of a motor neurone disease that can be treated. Alzheimer's disease is an example of a neurodegenerative disease that can be treated. Examples of mental disorders that can be treated by one or more of the peptides include psychosis and neurosis. For example, the peptides may be used to treat emotional disturbances, especially the emotional disturbances of psychiatric patients in a state of depression. The peptides may also be used as an auxiliary withdrawal treatment for drug addicts, after a period of detoxification, and in persons dependent on stimulants.

In another advantageous embodiment of the invention, one or more of peptides A-1 to D-1 may be used in the treatment of disorders of the immune system, particularly chronic disorders of the immune system the may occur spontaneously in people of advanced age. The peptides can also be used in the treatment of diseases requiring immune-modulation. The peptides are useful in the treatment of a variety of diseases with an immunological and infectious basis. For example, they can be used to treat chronic diseases with a bacterial and viral aetiology, and to treat acquired immunological deficiencies that have developed, for example, after chemotherapy or radiotherapy of neoplasms. The peptides may be used for treating chronic bacterial and viral infections requiring non-specific immunostimulation and immunocorrection.

A chronic disorder is a disorder that has persisted, or is expected to persist, for a long time, i.e., at least 3 months and usually at least 6 months.

One or more of the peptides may be used for improving the development of the immune system of a new born child. It is a further feature of the invention to use the peptides to correct immunological deficiencies in a child. These uses of the peptides may be particularly applicable to babies or children who have been deprived of colostrum. This may occur, for example, in babies and children who were not breast fed from birth.

The peptides, either alone or in combination with one another, also have diagnostic and research applications. For example, the synthetic peptides, as well as the corresponding antibodies described below, may be used to recognise pathological processes occurring in a host. These processes may be induced by excessive production or inhibition of the peptides or the antibodies. Once the pathological process associated with a particular level of the peptides or the antibodies is known, measuring the production of the peptides and the antibodies in body fluids may be used to determine pathological processes taking place in the host.

According to another aspect of the invention, we provide the use of one or more of peptides A-1 to D-1 as a dietary supplement. This dietary supplement is particularly useful for babies, especially premature babies and babies at term, and for young children to correct deficiencies in the development of their immune system. The dietary supplement may also be used as a dietary supplement for adults, including senile persons, who have been subjected to chemotherapy, or have suffered from cahexia, or weight loss due to chronic disease.

In an aspect of the invention, we provide a dietary supplement comprising an orally ingestible combination of one or more of peptides A-1 to D-1 in combination with a physiologically acceptable carrier. The dietary supplement may be provided in liquid or solid form; the dietary supplement may suitably be provided in the form of a tablet. The dietary supplement may be provided in the form of a baby food formula. The dietary supplement may include, as an additive, lactoferrin and/or selenium and/or a group of cytokines containing members of the interferon family.

In accordance with the invention, one or more of peptides A-1 to D-1 may be administered prophylactically in order to help to prevent the development of disorders of the central nervous system and the immune system.

The peptides according to the invention may be used to promote the dissolution of β-amyloid plaques, and, therefore, the peptides may be used in the treatment of any disease which is characterised by the development of β-amyloid plaques.

The peptides according to the invention may be administered in a dosage in the range 1 ng to 10 mg. A dosage unit of about 3 μg is typical. However, the optimum dosage will, of course, depend upon the condition being treated.

The peptides according to the invention may be formulated for administration in any suitable form. Thus, the invention further provides a composition, especially a pharmaceutical composition, which includes one or more of the peptides in combination with a physiologically acceptable carrier. The peptides may, for example, be formulated for oral, topical, rectal or parenteral administration. More specifically, the peptides may be formulated for administration by injection, or, preferably, in a form suitable for absorption through the mucosa of the oral/nasopharyngeal cavity, the alimentary canal or any other mucosal surface. The peptides may be formulated for administration intravenously, subcutaneously, or intramuscularly. The oral formulations may be provided in a form for swallowing or, preferably, in a form for dissolving in the saliva, whereby the formulation can be absorbed in the mucous membranes of the oral/nasopharyngeal cavity. The oral formulations may be in the form of a tablet for oral administration, lozenges (i.e. a sweet-like, tablet in a form suitable to be retained in the mouth and sucked), or adhesive gels for rubbing into the gum. The peptides may be formulated as an adhesive plaster or patch, which may be applied to the gums. The peptides may also be formulated for application to mucous-membranes of the genitourinary organs. The topical formulations may be provided in the form of, for example, a cream or a gel.

One or more of the peptides may be incorporated into products like milk or cheese spread.

According to another aspect of the invention there is provided a pharmaceutical composition comprising a peptide containing the amino acid chain LQTPQPLLQVMMEPQGD; DPPPPQS; and/or LFFFLPVVNVLP or use as an immunosuppressant, for use in the treatment of autoimmune disorder, and/or for use in suppressing the rejection of transplanting organs. The invention also embraces the use of one or more of these peptides in the manufacture of a medicament for use as an immunosuppressant, for use in the treatment of autoimmune disorder, and/or for use in suppressing the rejection of transplanting organs.

We have found that the ratio of the peptides in colostrum varies over time. Owing to hormonal changes, many proteins secreted into colostrum become sequentially degraded. The longer the time from parturition the more extensive the degradation can be. This knowledge will help with the design of new baby food formulas as well as many drugs for immuno-compromised patients.

In another aspect, the invention provides an antibody for each of the peptides A-1 to D-1, and provides compositions containing said antibodies. In particular the invention provides the antibodies in substantially isolated form. The antibodies can be produced by injecting a suitable mammalian subject, such as a rabbit, with the corresponding peptide (with a suitable adjuvant), then recovering the antibodies from the subject after allowing time for them to be produced. This technique is described in detail in Example 3. It is possible to test that the correct antibody has been produced by ELISA (enzyme-linked immunosorbent assay) using the synthetic peptides as antigens. The antibodies can be further tested against the natural peptides in Colostrinin as confirmation that the synthetic peptides do correspond to the natural peptides found in Colostrinin. The antibodies have potential uses in therapy, as a diagnostic tool and as a research tool.

The invention also encompasses the selective administration of one or more of peptides A-1 to D-1, at selected times to a patient, and the selective administration of one or more of the antibodies for the peptides in order to switch on or off the activity of the peptides at a selected time.

A selection of selected ones of the peptides and/or antibodies may be provided in a single composition which is specially tailored to produce a particular effect. For example, for a person with an immunological disorder, the composition can be specially tailored for that disorder. The composition may be specially selected for more than one disorder. The composition may be specially selected to restore or produce a particular balance in a subject.

In some applications it may be desirable to provide a pharmaceutical composition which contains one or more of the peptides and one or more of the antibodies in combination with a physiologically acceptable carrier.

The invention further embraces the use of one or more of the peptides and/or antibodies in the manufacture of a medicament for use in any of the therapeutic applications described above.

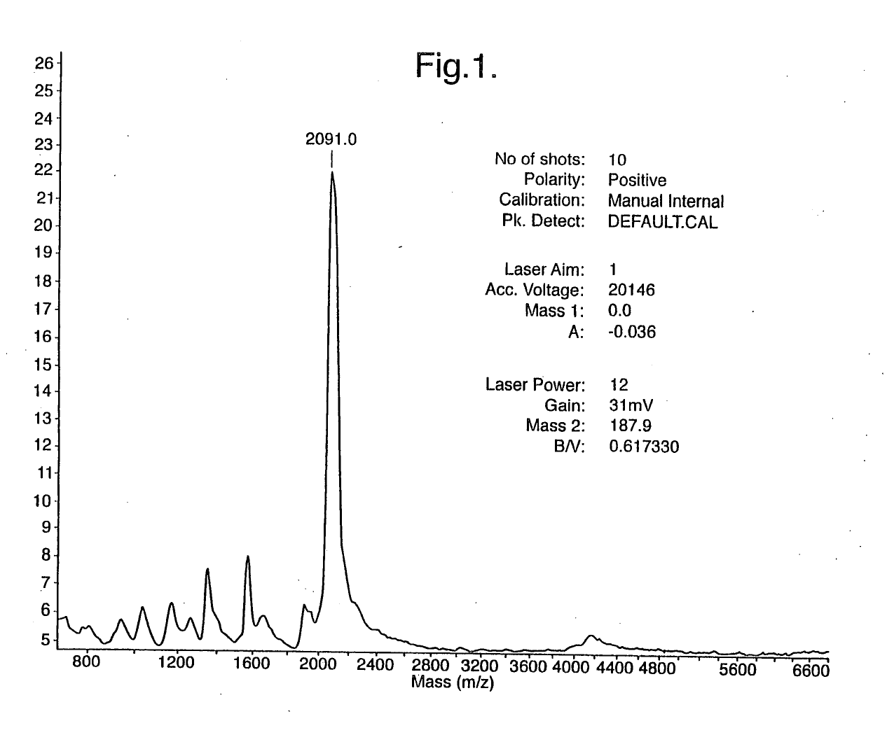

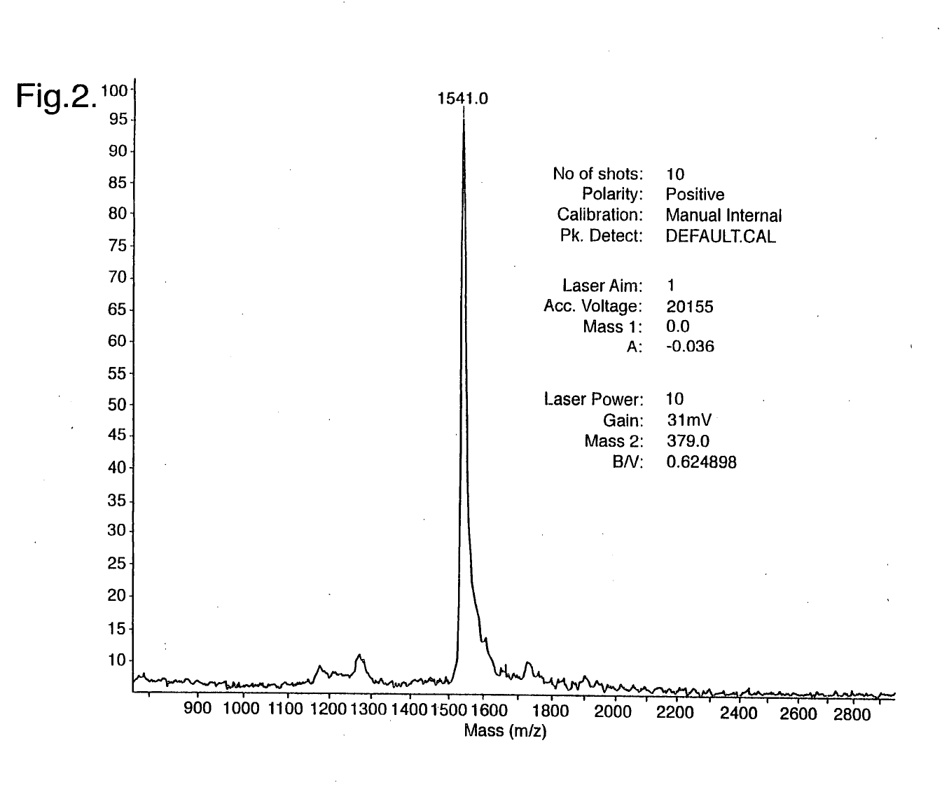

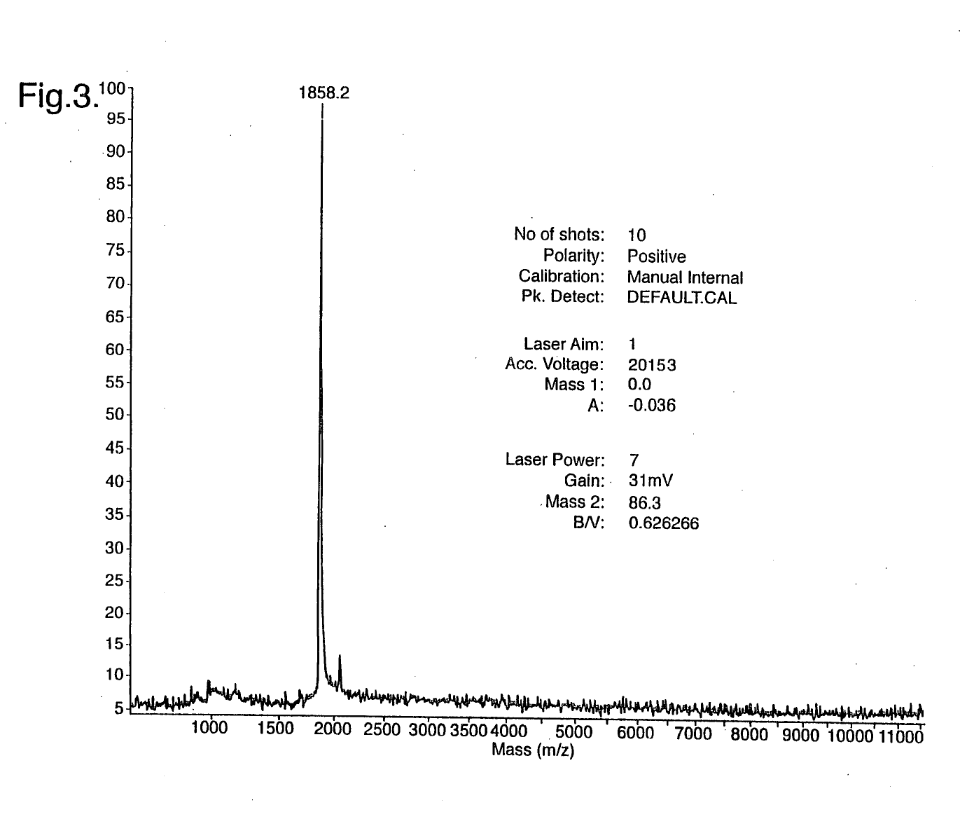

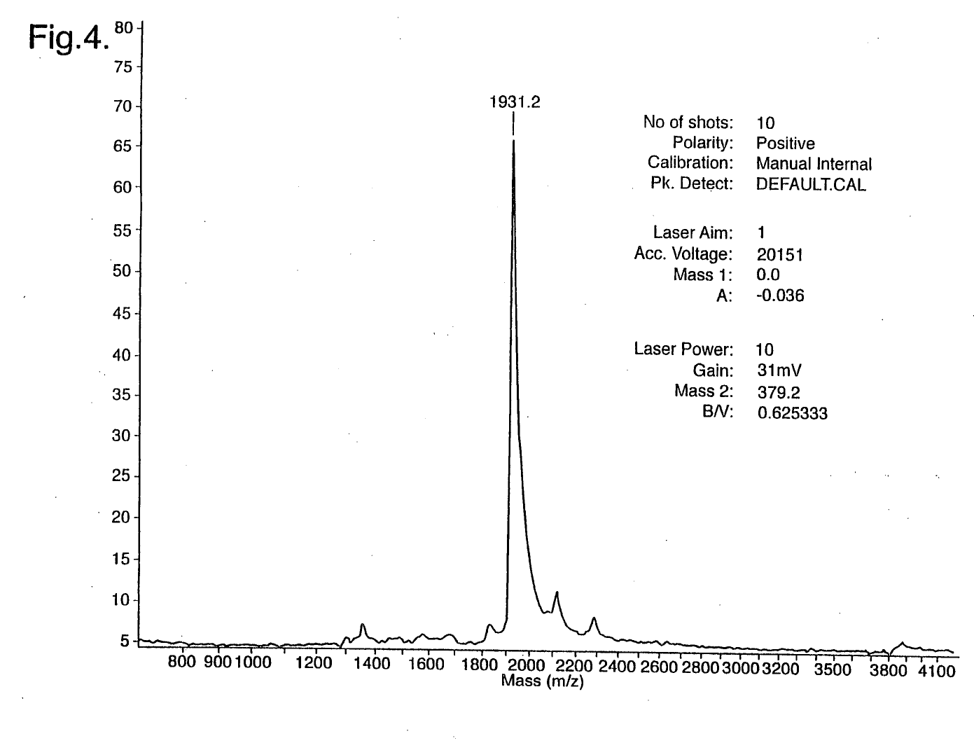

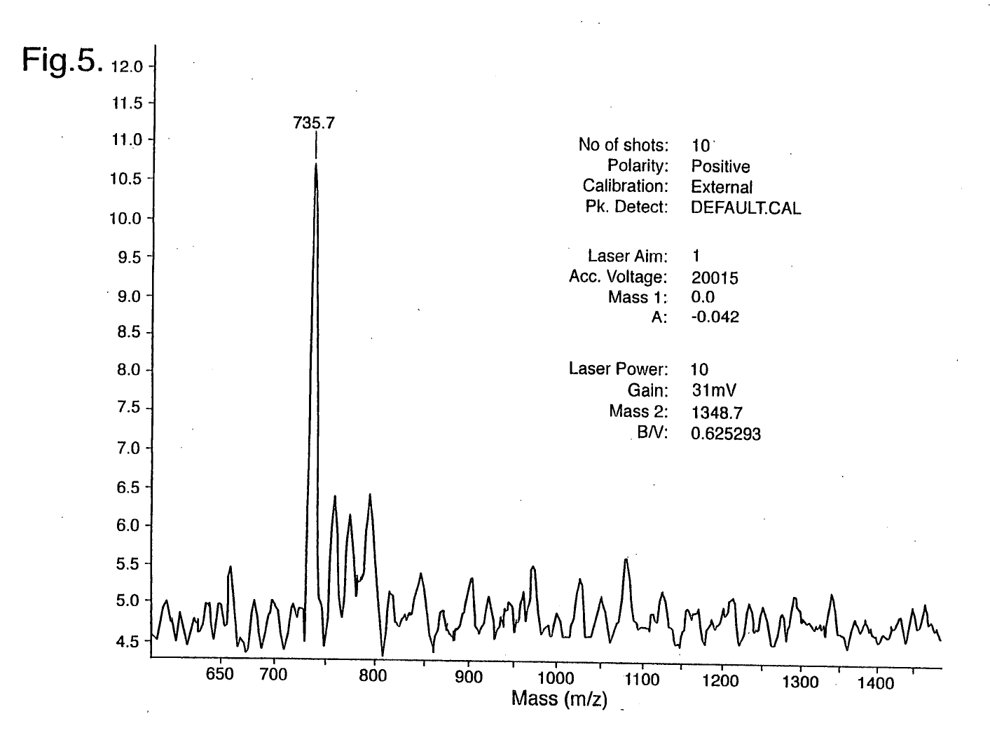

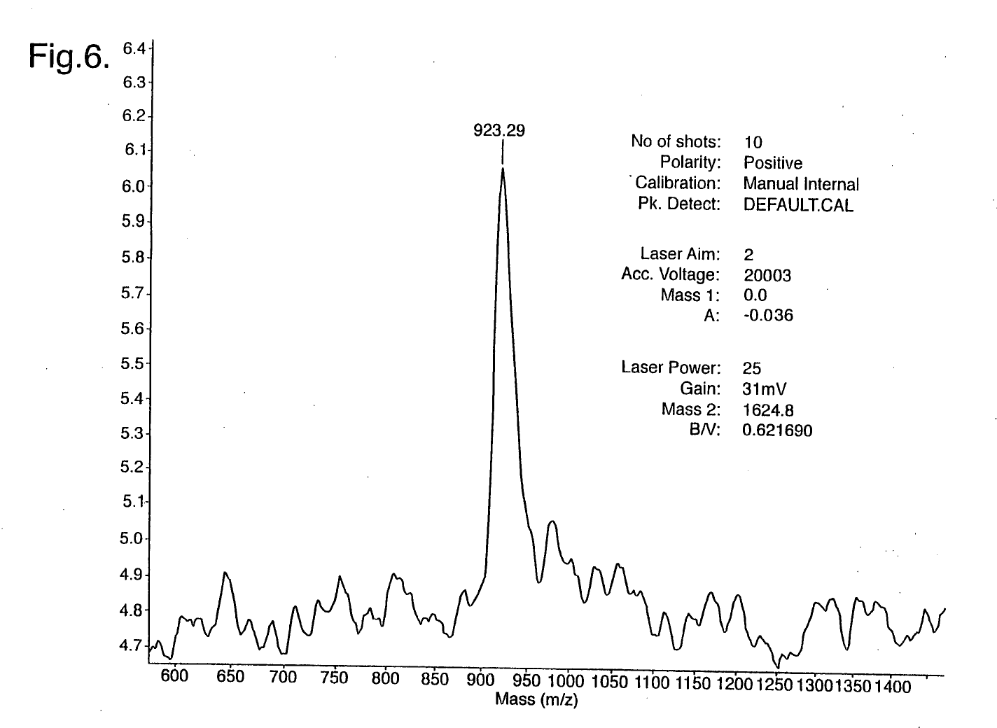

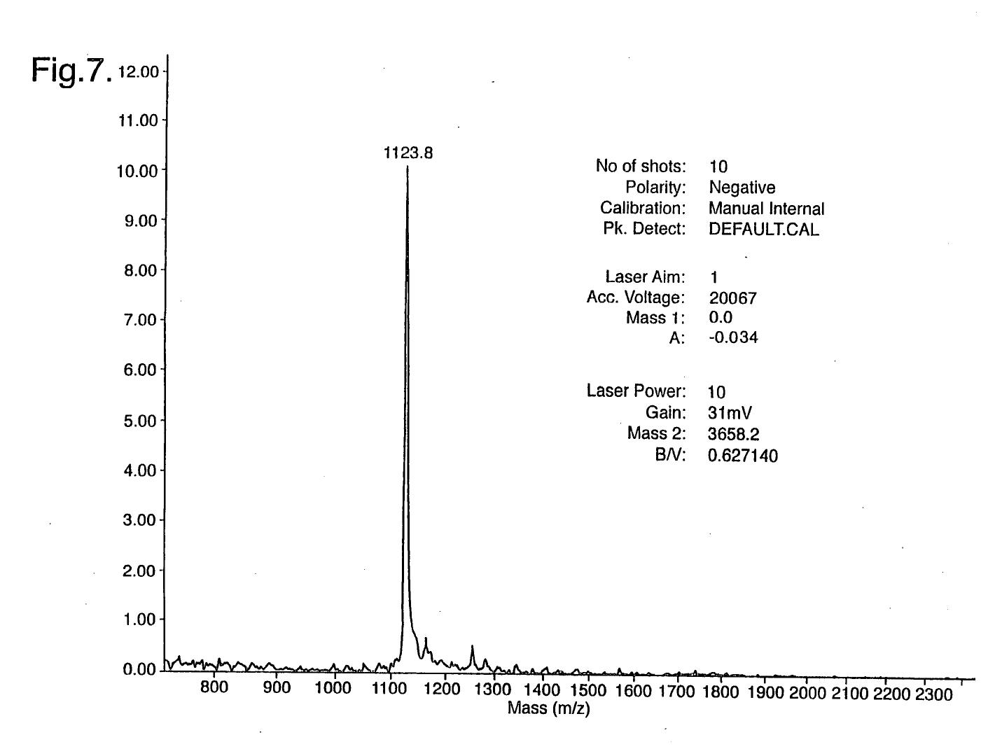

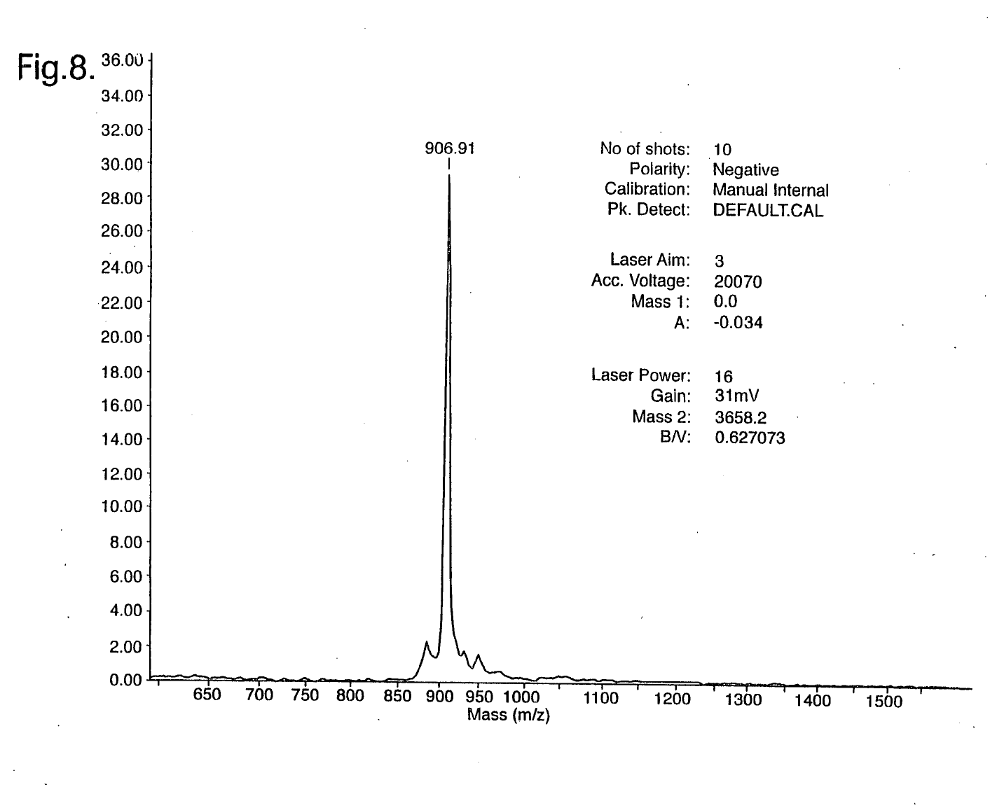

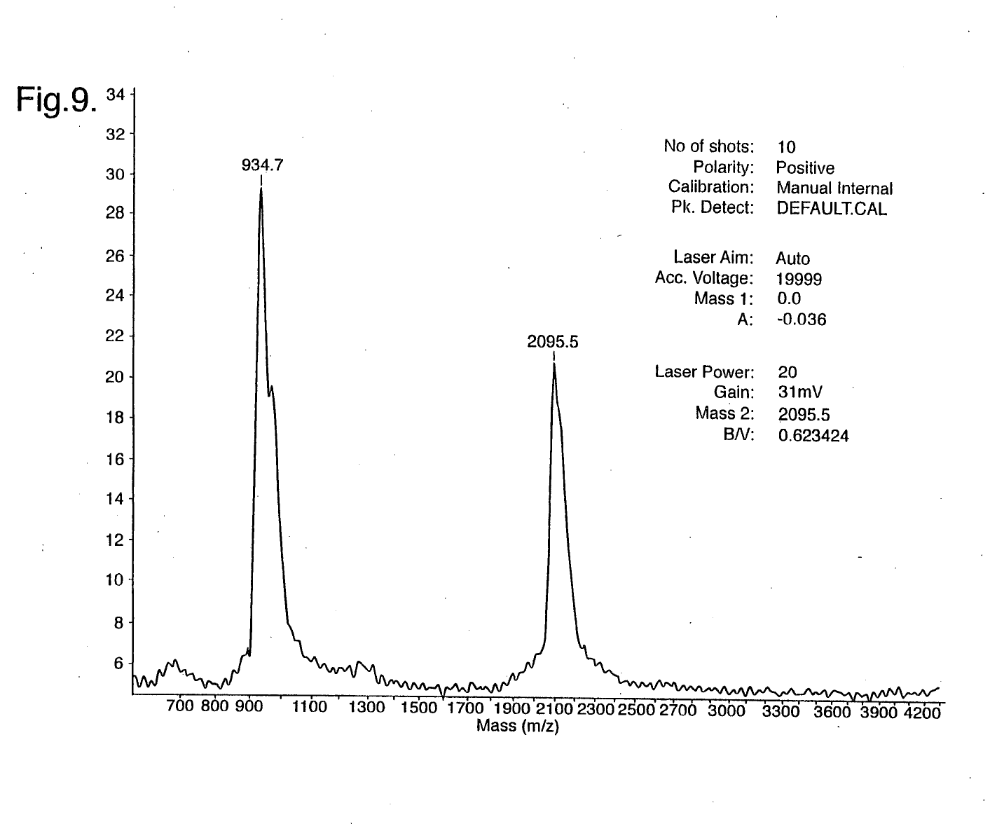

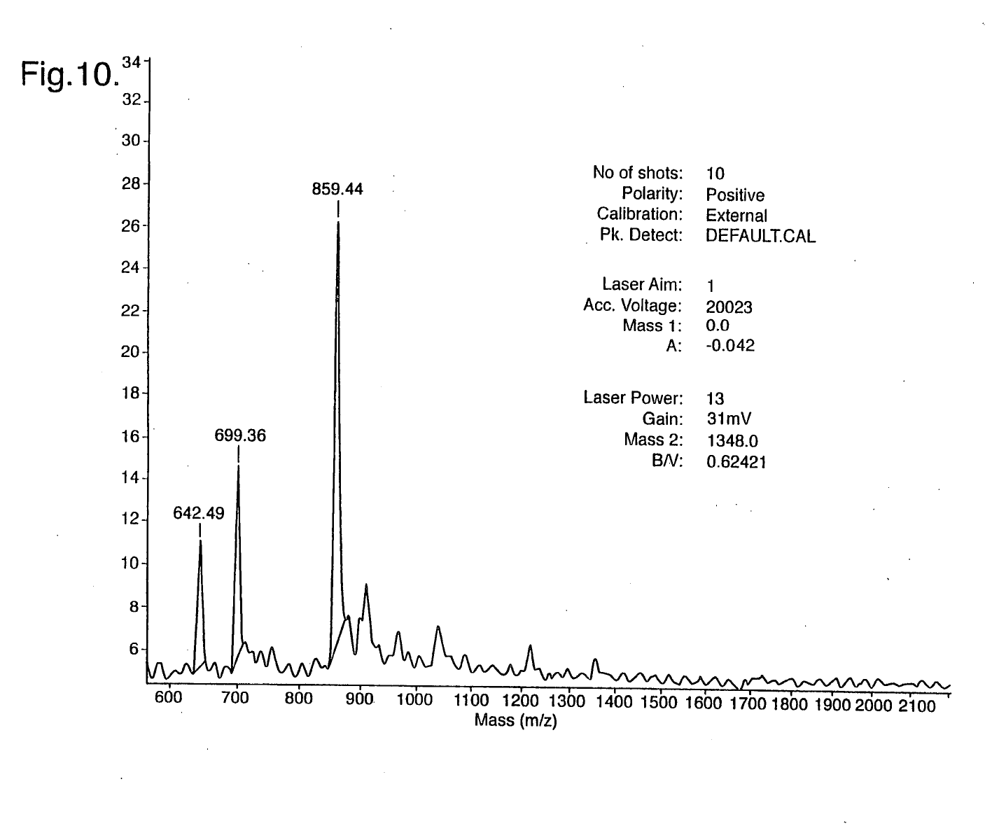

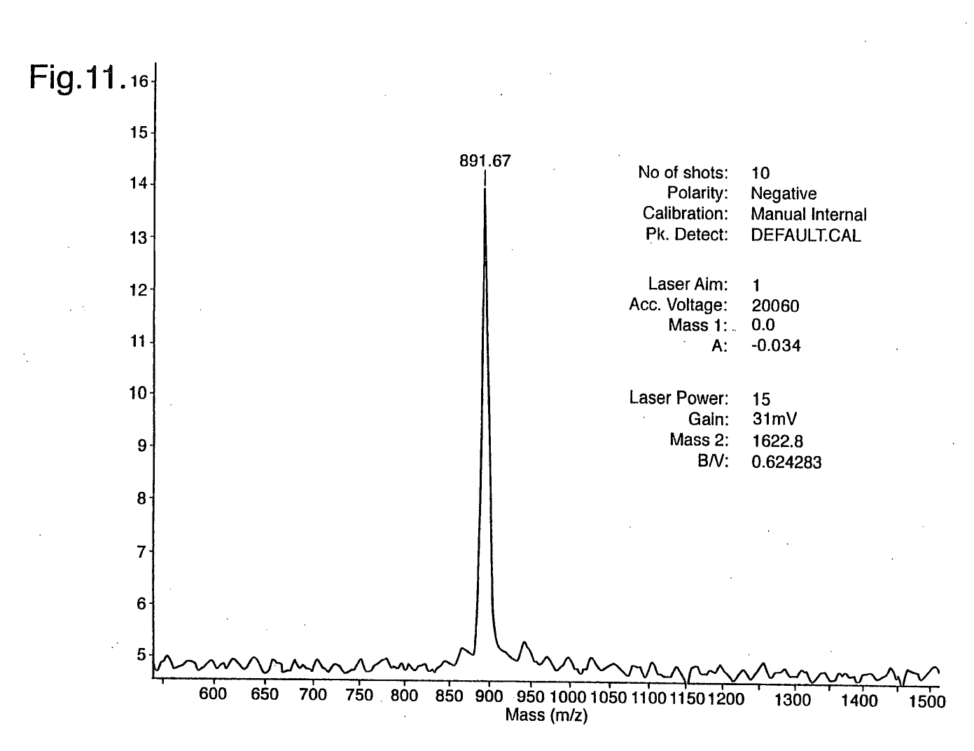

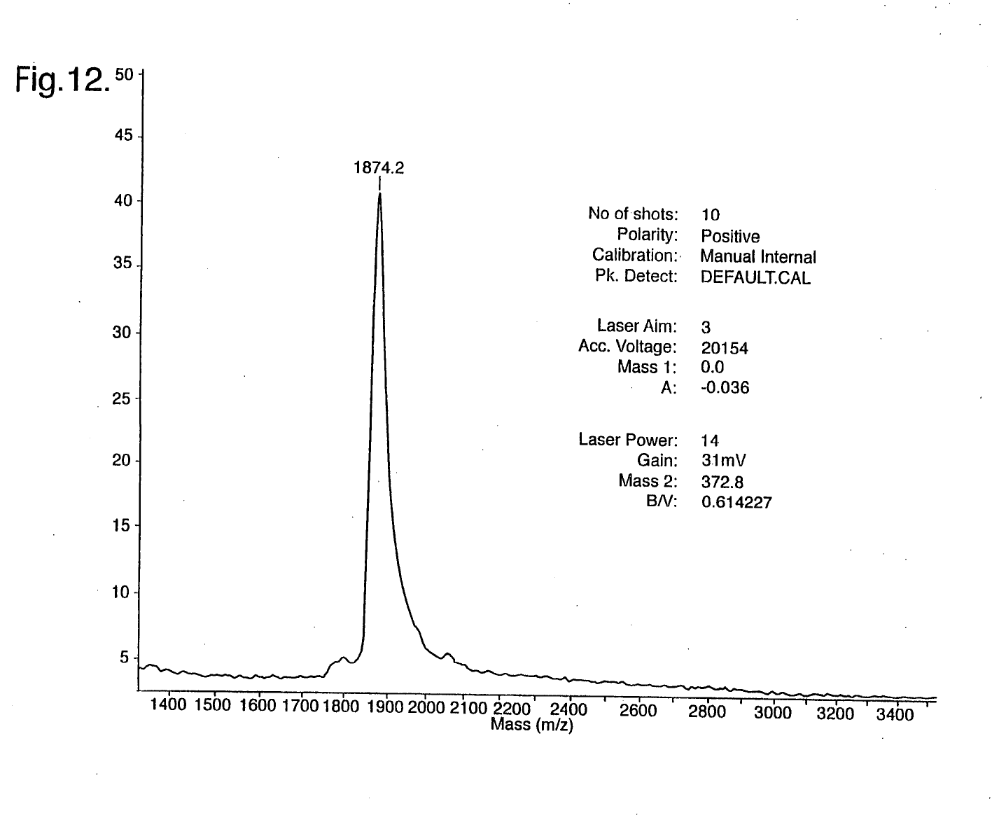

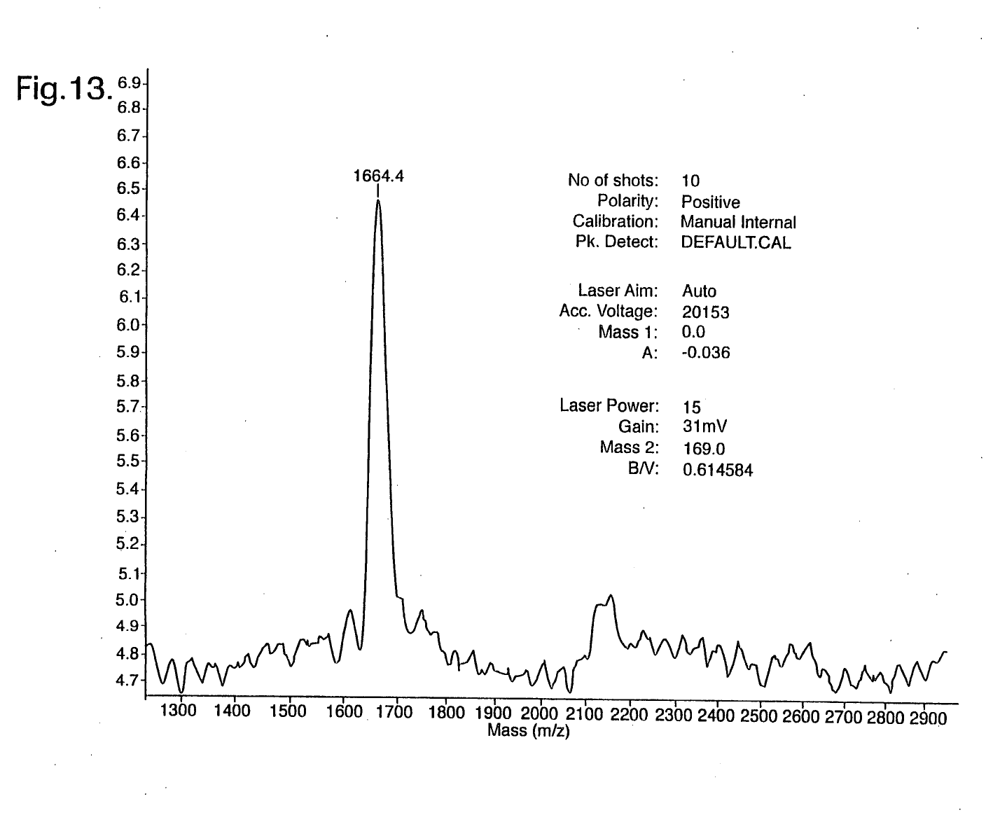

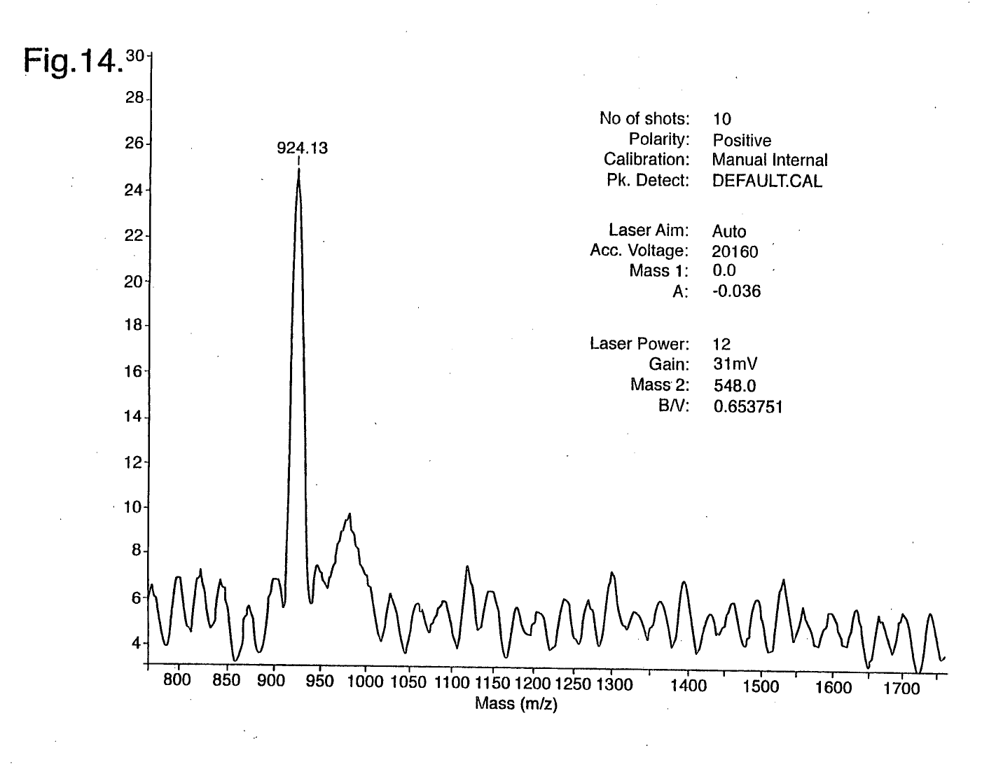

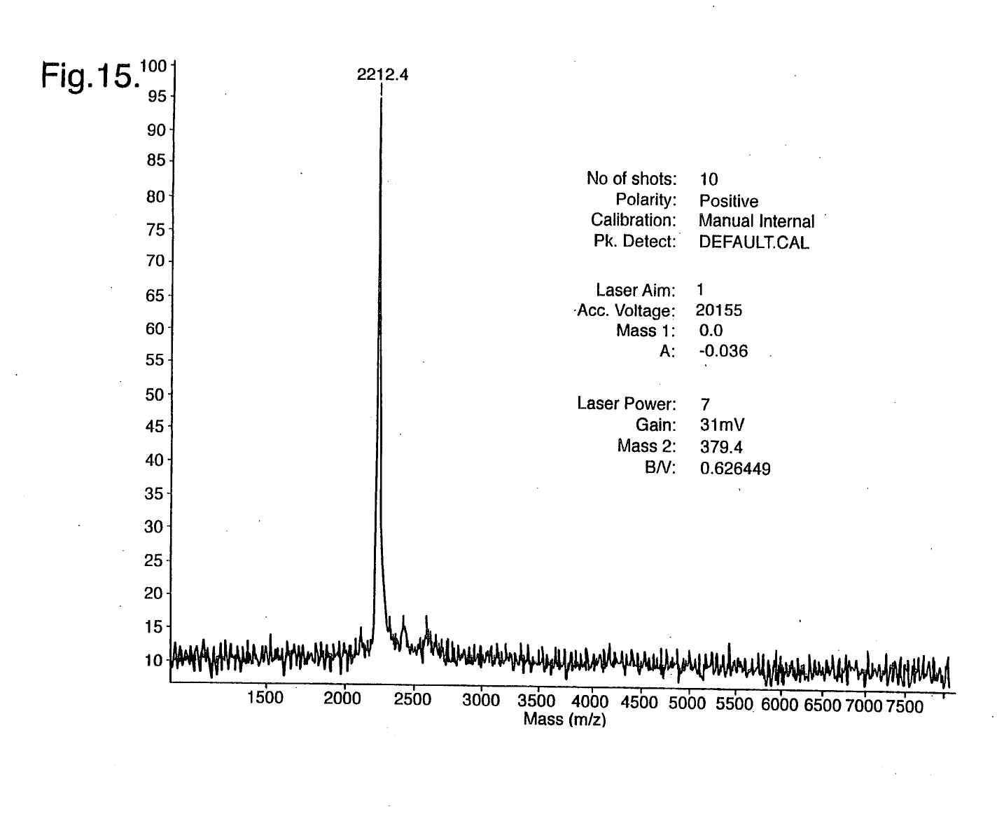

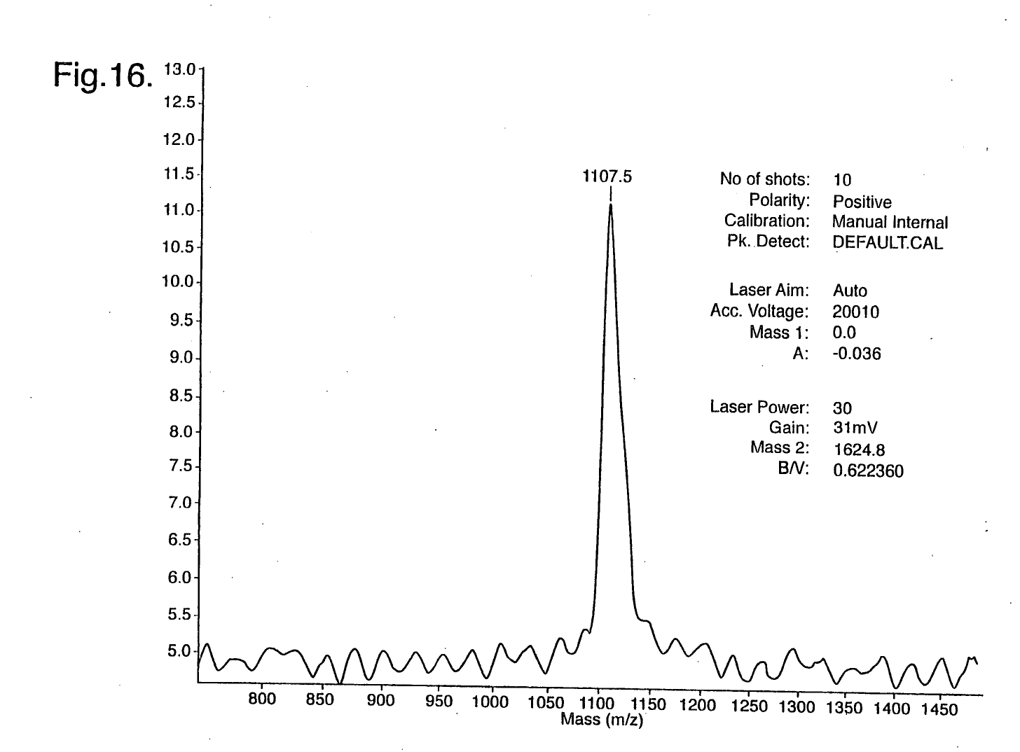

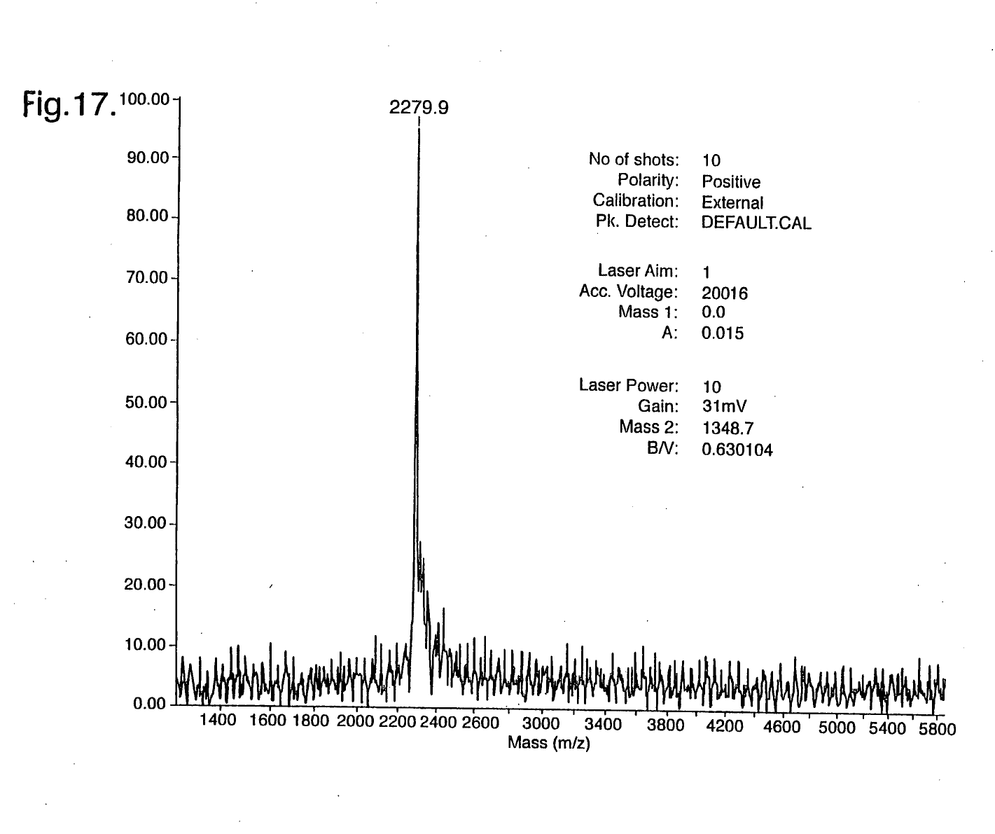

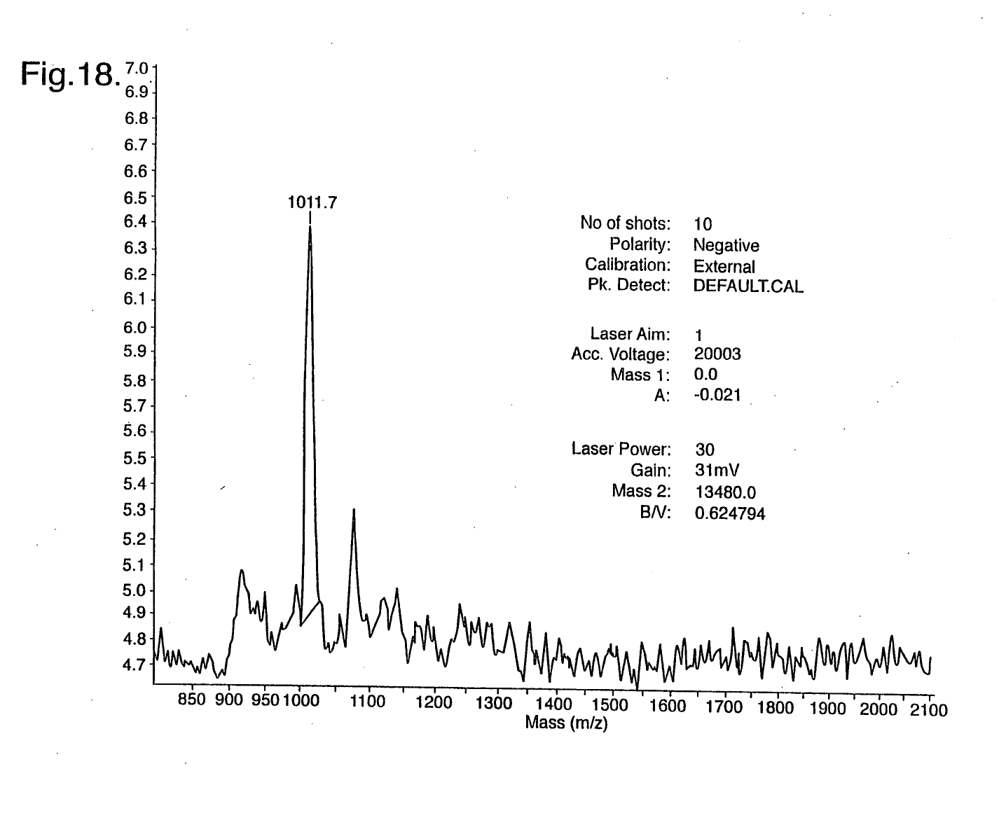

Reference is now made to the accompanying drawing in which FIGS. 1 to 18 are Matrix-Assisted Laser Desorption Time-of-Flight Mass Spectroscopy (LDMS) spectra of certain peptides according to the invention.

The invention will now be further described with reference to the following examples.

### EXAMPLE 1

Preparation of Colostrinin

Colostrinin can be prepared by techniques already disclosed in the prior art, including, for example, WO-A-98/14473. Colostrum collected from the ewe within 12 hours post parturition can be purified by centrifuging to eliminate cellular and lipidic components, pH shifting to eliminate nutritional components, ammonium sulfate precipitation, ion exchange chromatography and molecular sieving.

### EXAMPLE 2

Identification of the Components of Colostrinin

Initially the Colostrinin produced according to example 1 was analysed by SDSPAGE, by means of which we found the following two peptides: VLEMKFPPPPQETVT (A-3) and LKPFKLKVEVFPEP (A-4). However, we could not identify any other peptides with this technique, so we turned to hplc.

The Colostrinin produced in example 1 was fractionated by hplc using a C-18 reverse-phase column. This technique was used to separate the peptides exhibiting different hydrophobic patterns, present in Colostrinin. The hplc column was obtained from Separation Methods Technologies (who are based in Newark, Del., U.S.A). The column type was designated C-18 and was 150 mm in length by 10 mm in diameter. The column was packed with particles having a particle size of 3 μm having a pore size of 30 nm. The pump module and diode array were supplied by Beckman (who are based in Fullerton, Calif., U.S.A.): a Beckman System Gold 126 pump module was used, and a Beckman System Gold 168 diode array detector module was used.

The Colostrinin was loaded in 0.1% trifluoroacetic acid (TFA) dissolved in hplc grade water. A 500 μl sample, containing approximately 900 picomole of the Colostrinin was loaded on the column, the column having been equilibrated prior to loading. After approximately 10 minutes of intensive washing, the material was eluted by gradient formed from solutions A and B, under a regime indicated in Table 1. During this time, the flowrate through the column was 0.06 ml/min.

TABLE 1

| Time/Min | % Solvent A | % Solvent B |
|---|---|---|
| 0.00 | 95.0 | 5.0 |
| 10.00 | 30.0 | 70.0 |
| 100.00 | 0.0 | 100.0 |
| 140.00 | 95.00 | 5.0 |
| 150.00 | 95.9 | 5.0 |

Solvent A: 0.1% TFA (trifluoroacetic acid) in hplc grade water.
Solvent B: 70% acetonitrile fluoride and 0.09% TFA in hplc grade water.

The peptides found at the peaks in the hplc were then individually analysed using Edman Degradation; this was done using a Beckman LF3000 sequencer. Each concentrated fraction was loaded into a pre-salted Beckman peptide support disk. The samples were sequenced using the standard Edman degradation steps. Typically, 10 to 100 pmoles were used to generate 10 to 25 cycles for each analysis.

Subsequently, each fraction was analysed by the Inline hplc System. This used a Hewlett Packard PTH-M column having a length of 250 mm and a diameter of 2.1 mm. The Beckman System Gold 126 pump module was used, and the Beckman System Gold 168 diode array detector module was used. The flowrate in the column was 0.275 ml/min, and the solvent composition was varied as shown in Table 2.

TABLE 2

| Time/Min | % Solvent A | % Solvent B |
|---|---|---|
| 0.00 | 80.0 | 20.0 |
| 0.10 | 62.0 | 38.0 |
| 17.10 | 10.0 | 90.0 |
| 28.10 | 87.5 | 12.5 |

Solvent A: 3.5% THF (tetrahydrofuran), 1.5% acetonitrile fluoride premix, 1% acetic acid & 0.02% TEA (triethanolamine) in hlpc grade water.
Solvent B: 12% isopropanol. in acetonitrite.

The structure of the peptides A-1 to D-1 was then used for comparative studies with sequences registered in two known computer programs: Wu-Blast 2 of the National Center for Biotechnology Information NR Protein Data Base; and Beauty—Post Processing provided by the Human Genome Center, Baylor College of Medicine, Houston, Tex., USA. This made is possible to determine whether any of the peptide sequences P1-P32 were already known.

The results of the Edman degradation are summarised in Table 3. The subsequent analysis with the computer programs revealed that there were at least two different precursor proteins for the peptides in Colostrinin: β-casein and annexin. Furthermore, by using the Tremble program, it was possible to find evidence that some of the peptides may have a precursor which is a casein homologue. Finally, some of the peptides had unique sequences with no homology to any known protein.

TABLE 3

| | | | AA sequence | | |
|---|---|---|---|---|---|
| Peak | Elution | | | Unknown | |
| No. | time min. | Area % | Casein homologue | precursor | Casein/Annexin precursor |
| 1 | 8.54 | 1.181 | VVMEV (B-6) | | ATFNRYQDDHGEEILKSL |
| | | | | | (D-1) |
| 2 | 29.086 | 0.124 | | SEQP (A-5) | |
| 3 | 53.775 | 0.579 | | | |
| 4 | 56.815 | 0.111 | FPPPK (B-5) | | LSQPKVLPVPQKAPQPRDMPIQ |
| | | | | | (C-11) |
| 5 | 58.044 | 2.101 | DSQPPV (B-4) | | LSQPKVLPVPQKAPQPRDMPIQ |
| | | | | | (C-11) |
| 6 | 60.488 | 0.588 | MQPPPLP (B-9) | | LSQPKVLPVPQKAPQPRDMPIQ |
| | | | | | (C-11) |
| 7 | 62.684 | 1.273 | | DPPPPQS (A- | |
| | | | | 7) | |
| 8 | 65.44 | 3.247 | | LQTPQPLLQVMMEPQGD | LSQPKVLPVPQKAPQPRDMPIQ |
| | | | | (A-1) | (C-11) |
| 9 | 66.775 | 0.683 | DQPPDVEKPDLQPFQVQS | | LSQPKVLPVPQKAPQPRDMPIQ |
| | | | (B-10) | | (C-11) |
| 10 | 67.929 | 2.943 | LFFFLPVVNVLP | | LSQPKVLPVPQKAPQPRDMPIQ |
| | | | (B-8) | | (C-11) |
| | | | | | MHQPPQPLPPTVMFP (C-9) |
| 11 | 69.229 | 2.717 | SEEMP (B-3) | | LSQPKVLPVPQKAPQPRDMPIQ |
| | | | | | (C-11) |
| | | | | | HKEMPFPKYPVEPFTESQ |
| | | | | | (C-5) |
| 12 | 70.984 | 2.964 | KYKLQPE (B-2) | | LSQPKVLPVPQKAPQPRDMPIQ |
| | | | | | (C-11) |
| | | | | | HKEMPFPKYPVEPFTESQ |
| | | | | | (C-5) |
| 13 | 72.547 | 1.423 | VLPPNVG (B-1) | | LSQPKVLPVPQKAPQPRDMPIQ |
| | | | | | (C-11) |
| 14 | 74.09 | 1.425 | DLEMPVLPVEPFPFV | | SLPQNILPL (C-2) |
| | | | (B-7) | | |
| 15 | 76.558 | 5.268 | | MPQNFYKLPQM | MHQPPQPLPPTVMFP (C-9) |
| | | | | (A-2) | |
| 16 | 78.506 | 6.978 | | LNF (A-8) | MHQPPQPLPPTVMFP (C-9) |
| 17 | 80.94 | 4.224 | | | MHQPPQPLPPTVMFP (C-9) |
| | | | | | SLTLTDVEKLHLPLPLVQ |
| | | | | | (C-6) |
| | | | | | PQSVLS (C-9) |
| 18 | 83.8 | 1.025 | | | ND |
| 19 | 84.314 | 2.151 | | | MHQPPQPLPPTVMFP (C-9) |
| 20 | 85.707 | 3.103 | | | SWMHQPP (C7) |
| 21 | 87.061 | 1.047 | | | ND |
| 22 | 87.907 | 1.529 | | | ND |
| 23 | 88.921 | 1.311 | | | MHQPPQPLPPTVMFP (C-9) |
| | | | | | SLTLTDVEKLHLPLPLVQ |
| | | | | | (C-6) |
| | | | | | TQTPVVVPPF (C-3) |
| | | | | | VYPFTGPIPN (C-1) |
| 24 | 89.856 | 1.114 | | | ND |
| 25 | 91.343 | 0.906 | | | ND |
| 26 | 92.667 | 0.821 | | | ND |
| 27 | 93.521 | 3.893 | | | ND |
| 28 | 94.751 | 1.426 | | | ND |
| 29 | 95.82 | 0.272 | | | HKEMPFPKYPVEPFTESQ |
| | | | | | (C-5) |
| 30 | 96.697 | 3.164 | | | QPLPPTVMFP (C-8) |
| | | | | | HKEMPFPKYPVEPFTESQ |
| | | | | | (C-5) |
| 31 | 97.938 | 3.266 | | | ND |
| 32 | 99.893 | 5.621 | | | HKEMPFPKYPVEPFTESQ |
| | | | | | (C-5) |
| 33 | 100.9 | 5.032 | | | ND |
| 34 | 102.709 | 4.007 | | | AFLLYQE (C-12) |
| | | | | | HKEMPFPKYPVEPFTESQ |
| | | | | | (C-5) |
| 35 | 104.74 | 3.275 | | | ND |
| 36 | 106.01 | 2.231 | | | ND |
| 37 | 170.75 | 3.037 | | | ND |
| 38 | 108.782 | 2.173 | | | SLTLTDVEKLHLPLPLVQ |
| | | | | | (C-6) |
| | | | | | HKEMPFPKYPVEPFTESQ |
| | | | | | (C-5) |
| | | | | | SLPQNILPL (C-2) |
| | | | | | VYPFTGPIPN (C-1) |
| 39 | 111.056 | 5.375 | | | HKEMPFPKYPVEPFTESQ |
| | | | | | (C-5) |
| 40 | 112.679 | 1.901 | | | ND |
| 41 | 114.707 | 0.436 | | | ND |
| 42 | 8.54 | 1.181 | | | ATFNRYQDDHGEEILKSL |
| | | | | | (D-1) |

ND indicates that these fractions were not analysed.

DKE (A-6), LQPEIMGVPKVKETMVPK (C4), FLLYQEPVLGPVR (C-11) and RGPFPILV (C-13) were also detected by hplc, although their presence is not indicated in the above table.

### EXAMPLE 3

Production of the Antibodies

The peptides identified in example 2 were produced by the synthetic technique known as the solid phase method. This method involved the following steps:
1. Wash pre-loaded resin with DMF (dimethylformamide), then drain completely.
2. Add 10 ml of 20% piperidine/DMF to resin. Shake for 5 mins, then drain.
3. Add another 10 ml of 20% piperidine/DMF. Shake for 30 mins.
4. Drain reaction vessel and wash resin with DMF four times. Then wash once with DCM (dichloromethanol). Check beads using the ninhydrin test—the beads should be blue.
5. The coupling step was carried out as follows:
   Prepare the following solution:
      1 mmole Fmoc (i.e. fluorenylmethyloxycarbonyl) amino acid 2.1 ml of 0.45 M HBTU/HOST (1 mmol) (2-(1H-benzotriazol-1-yl)-1,1,3,3-tetramethyluronium hexafluorophosphatelN-hydroxybenzotriazole-H₂O)
      348 μl of DIEA (2 mmol) (diisopropylethylamine)
   Add the solution to the resin and shake for a minimum of 30 minutes.
6. Drain reaction vessel and wash the resin again with DMF four times and with DCM once.
7. Perform the ninhydrin test:
   If positive (no colour)—proceed to step 2 and continue synthesis.
   If negative (blue colour)—return to step 5 and recouple the same Fmoc amino acid.
8. After the synthesis was complete, the peptide was cleaved from the resin with 5% H₂O, 5% phenol, 3% Thionisole, 3% EDT (ethanedithiol), 3% triisopropylsilane and 81% TFA for 2 hours.
9. After 2 hours, filter into cold MTBE (methyl t-butyl ether). The precipitated peptide was then washed twice with cold MTBE and dried under nitrogen gas.
10. The molecular weight of the synthesised peptides was checked by Matrix-Assisted Laser Desorption Time-of-Flight Mass Spectroscopy (LDMS), and the purity was checked by hplc using a C-18, 300 Angstrom, 5 μm column. The resulting spectra of some peptides are shown in FIGS. 1 to 18.

To each N-terminal end of the synthetic peptides, L-cysteine was attached, and the peptide was formed into a ring so that the cysteine group lay between the N-terminal and the C-terminal ends of the synthetic peptide. This facilitated peptide conjugation with Keyhole Hemolymph (KHL). The shorter peptides (i.e. those containing 9 or fewer amino acids) were artificially elongated with biologically inert amino acids prior to attaching the L-cysteine. This was done in order to facilitate annealing and increase the antigenicity of the shorter peptides.

Table 4 shows a number of the peptides that were formed and indicates the figure number of the drawings which illustrates the laser desorption mass spectrum.

TABLE 4

| | ORIGINAL | |
|---|---|---|
| PEPTIDE SYNTHESIZED | PEPTIDE | FIG. NO. |
| NH₂—(Ac)CLQTPQPLLQVMMEPQGD-OH | A-1 | 1 |
| NH₂—(Ac)CMPQNFYKLPQM-OH | A-2 | 2 |
| NH₂—(Ac)CVLEMKFPPPPQETVT-OH | A-3 | 3 |
| NH₂—(Ac)CLKPFPKLKVEVFPFP-OH | A-4 | 4 |
| NH₂-SEQPGGGC-OH | A-5 | 5 |
| NH₂—(Ac)CGVLPPNVG-OH | B-1 | 6 |
| NH₂—(Ac)CGGGKYKLQE-OH | B-2 | 7 |
| NH₂—(Ac)CGGGSEEMP(amide)-OH | B-3 | 8 |
| NH₂—(Ac)CGGGDSQPPV-OH | B-4 | 9 |
| NH₂—CFPPPKGGGC-OH | B-5 | 10 |
| NH₂—(Ac)CGGGVVMEV-OH | B-6 | 11 |
| NH₂—(Ac)CDLEMPVLPVEPFPFV-OH | B-7 | 12 |
| NH₂—(Ac)CLFFFLPVVNVLPI-OH | B-8 | 13 |
| NH₂—(Ac)CMQPPPLP-OH | B-9 | 14 |
| NH₂—(Ac)CDQPPDVEKPDLQPFQVQS-OH | B-10 | 15 |
| NH₂—(Ac)CGAFLLYQE-OH | C-12 | 16 |
| NH₂—(Ac)CATFNRYQDDHGEEILKSL-OH | D-1 | 17 |
| NH₂-DPPPQSGGGC-OH | A-7 | 18 |

The invention further provides each of the peptides specified in Table 4, and the cyclisised version of each of these peptides, especially in isolated form and produced by a synthetic process. The term “Ac” represents an acyl group.

For immunisation, two young adult rabbits (5-6 months old, weighing 5-6 lbs [2.3-2.7 kg]) were used. Each antigen (i.e., each synthetic peptide) was given subcutaneously and intramuscularly in 0.1 ml injections at ten different sites. The protocol used followed the following sequence:

| Day | Procedure |
|---|---|
| 0 | Prebleed & initial inoculation of rabbit with 200 μg of the peptide |
| | at 0.5 ml of conjugate solution mixed with an equal volume of |
| | complete Freund's adjuvant (mineral oil/emulsifier/killed |
| | mycobacteria). |
| 14 | Boost inoculation with 200 μg of the peptide at 0.5 ml of |
| | conjugate solution mixed with an equal volume of incomplete |
| | Freund's adjuvant (mineral oil/emulsifier). |
| 28 | Boost (as on day 14) |
| | Production Bleed (approx. 20 ml sera) |
| 42 | Boost (as on day 14) |
| | Production Bleed (approx. 20 ml sera) |
| 56 | Boost (as on day 14) |
| | Production Bleed (approx. 20 ml sera) |
| 70 | Boost (as on day 14) |
| | Production Bleed (approx. 20 ml sera) |

This protocol may be varied. For example, the frequency of the production bleed depends upon, inter alia, the size and health of the host species.

The sera produced by this protocol were used for IgG purification on a Protein A matrix (from Sigma, based in St. Louis, Mo., USA). The protocol was as follows:
1. Wash columns with 10 ml 1×PBS (phosphate buffered saline). There were two 1 m column arranged in tandem each containing the Protein A matrix.
2. Add 3 ml of the serum to 3 ml of PBS and divide this mixture between the two Columns.
3. Collect the serum into a test tube as it drains through the column.
4. When the serum finishes draining, pour the washed serum back into the column and begin collecting flow through again. Repeat this step 5 to 6 times.
5. Wash the columns with 10 ml of 1×PBS.
6. Prepare several 1 ml tubes with 50 μl of 1 M TRIS (2-amino-2-hydroxymethyl-1,3-propanediol) (pH=9.5).
7. Add 1 ml of elution buffer (100 mM glycine, pH=2.8) to each tube and collect 1 ml of flow therethrough.
8. Move to the next prepare tube and repeat step 7.
9. Test each 1 ml sample by preparing ELISA plate with 10 μl of Bradford Assay and add 50 μl of each 1 ml flow through. Keep the samples that change the Bradford Assay from red to blue.
10. Dialyse the positive 1 ml samples together in 4 litres of 1×PBS at pH=7.2 for at least 24 hours.
11. Use spectrometer at 280 nm to find concentration of IgG in solution (extinction coefficient=1.4).
12. To store IgG solution, keep frozen at −4° C. to −20° C.

Table 5 shows the results for certain antibodies.

TABLE 5

| Peptide used | | | | | |
|---|---|---|---|---|---|
| to produce | Serum used | Purified Ab | | IgG | Total IgG |
| Antibody | (ml) | volume (ml) | OD₂₈₀ | (mg/ml) | (mg) |
| A-1 | 10 | 15 | 3.80 | 2.71 | 40.71 |
| A-2 | 10 | 15 | 2.13 | 1.52 | 22.82 |
| A-3 | 10 | 15 | 2.93 | 2.09 | 31.39 |
| A-4 | 10 | 15 | 3.57 | 2.55 | 38.25 |
| A-5 | 6 | 12 | 3.02 | 2.16 | 25.88 |
| B-1 | 10 | 15 | 2.64 | 1.89 | 28.28 |
| B-2 | 6 | 13 | 4.94 | 3.53 | 45.87 |
| B-3 | 6 | 13 | 5.01 | 3.58 | 46.52 |
| B-4 | 10 | 15 | 2.68 | 1.91 | 28.71 |
| B-5 | 10 | 15 | 2.28 | 1.63 | 24.43 |
| B-6 | 10 | 15 | 2.50 | 1.79 | 26.78 |
| B-7 | 10 | 15 | 2.90 | 2.07 | 31.07 |
| B-8 | 10 | 15 | 3.40 | 2.43 | 36.43 |
| B-9 | 10 | 15 | 3.80 | 2.71 | 40.71 |
| B-10 | 10 | 15 | 4.18 | 2.99 | 44.79 |
| C-12 | 10 | 15 | 1.95 | 1.39 | 20.89 |
| D-1 | 10 | 15 | 2.32 | 1.66 | 24.86 |
| A-7 | 6 | 12 | 3.33 | 2.38 | 28.54 |

The level of antibodies in the serum was established by ELISA (enzyme-linked immunosorbent assay) with the corresponding synthetic peptide antigen. This technique involved the following steps:
1. The antigen was diluted with a 0.1 M bicarbonate buffer (pH 9.0) to yield a 10 μg of antigen/ml solution. A volume of 50 μl of this solution was placed into each microwell of a 96 well plate.
2. The plates were covered and incubated at 37° C. for 3 hours.
3. The wells were washed with a coupling buffer and blocked using 200 μl of Pierce standard solution of BSA (bovine serum albumin).
4. 50 μl of dilutent BSA (0.75% soln.) was pipetted into each well. 50 μl of antibody serum sample diluted 1:100 in dilutent BSA were placed in lane A of each row.
5. 1:2 serial dilutions were performed moving down the plate.
6. The plates were covered and incubated at room temperature for 60 minutes.
7. The plates were washed four times with PBS wash solution.
8. A volume of 50 μl of goat anti-rabbit IgG (H&L) HRP conjugate at 1:1000 dilution in BSA was pipetted into each well and incubated at room temperature for 60 minutes (H&L=heavy and light chain; HRP=horseradish peroxidase).
9. The plates were washed four times with PBS wash solution.
10. A volume of 50 μl of substrate solution 2,2′-azino-bis(3-ethylbenzothiazoline-6-sulfonic acid)-diammonium salt (ABTS available from Pierce, which is used to help visualise the extent of the antibody/antigen reaction) was pipetted into each well and incubated at room temperature for about 2 minutes.
11. The reaction was stopped by adding 50 μl of 1% SDS (sodium dodecyl sulfate) into each well.
12. The wells were then read on a dynoplate reader at 405.

The data presented in Table 6 show the serum antibody titers against specific antibodies after the 10 week immunisation protocol.

TABLE 6

| | | Titre: (Serum Dilution) | | | |
|---|---|---|---|---|---|
| | | Pre Immu- | | Post | |
| | | nization | | Immunization | |
| No | Sequences | R1 | R2 | R1 | R2 |
| A. Peptides of unknown origin | | | | | |
| 1 | LQTPQPLLQVMMEPQGD | 0 | 0 | 6400 | 0 |
| 2 | MPQNFYKLPQM | 0 | 0 | 6400 | 25600 |
| 3 | VLEMKFPPPPQETVT | 0 | 0 | 6400 | 12800 |
| 4 | LKPFPKLKVEVFPFP | 0 | 0 | 6400 | 25600 |
| 5 | SEQP | 0 | 0 | 3200 | 25600 |
| 6 | DKE | ND | ND | ND | ND |
| 7 | DPPPPQS | 0 | 0 | 3400 | 6200 |
| 8 | LNF | ND | ND | ND | ND |
| B. Peptides from casein homologue | | | | | |
| 1 | VLPPNVG | 0 | 0 | 26500 | 25600 |
| 2 | KYKLQPE | 0 | 0 | 25600 | 25600 |
| 3 | SEEMP | 0 | 0 | 25600 | 12800 |
| 4 | DSQPPV | 0 | 0 | 25600 | 25600 |
| 5 | FPPPK | 0 | 0 | 12800 | 6400 |
| 6 | VVMEV | 0 | 0 | 25600 | 25600 |
| 7 | DLEMPVLPVEPFPFV | 0 | 0 | 25600 | 6400 |
| 8 | LFFFLPVVNVLP | 0 | 0 | 200 | 200 |
| 9 | MQPPPLP | 0 | 0 | 3200 | 12800 |
| 10 | DQPPDVEKPDLQPFQVQS | 0 | 0 | 12800 | 25600 |
| C. Peptides from β-casein | | | | | |
| 1 | VYPFTGPIPN | ND | 0 | ND | >10000 |
| 2 | SLPQNILPL | ND | 0 | ND | >10000 |
| 3 | TQTPVWPPF | ND | 0 | ND | >10000 |
| 4 | LQPEIMGVPKVKEMVPK | ND | 0 | ND | >10000 |
| 5 | HKEMPFPKYPVEPFTESQ | ND | 0 | ND | >10000 |
| 6 | SLTLTDVEKLHLPLPLVQ | ND | ND | ND | >10000 |
| 7 | SWMHQPP | ND | ND | ND | ND |
| 8 | QPLPPTVMFP | ND | 0 | ND | ND |
| 9 | MHQPPQPLPPTVMFP | ND | 0 | ND | >10000 |
| 10 | PQSVLS | ND | ND | ND | ND |
| 11 | LSQPKVLPVPQKAVPQRDMPIQ | ND | 0 | ND | >10000 |
| 12 | AFLLYQE | ND | 0 | 12800 | 25600 |
| 13 | FLLYQEPVLGPVR | ND | 0 | ND | >10000 |
| 14 | RGPFPILV | ND | ND | ND | ND |
| D. Peptide from annexin | | | | | |
| 1 | ATFNRYQDDHGEEILKSL | 0 | 0 | 12800 | 25600 |

ND = Not Done

In Table 6 the results are shown for two rabbits R1 and R2. In general, these results indicate that the potency of the antibodies produced in respect of peptides was excellent, and therefore that each antibody was the correct antibody for its synthetic peptide antigen. The antibodies produced by this technique were monospecific. However, the antigenic response in respect of peptides A-1, A-7 and B-8 were significantly lower than expected and lead us to predict that these peptides, especially B-8, would be useful as an immunosuppressant, and therefore would be useful in the treatment of autoimmune disorder and in the prevention of organ rejection during, for example, organ transplants.

### EXAMPLE 4

In order to establish that the peptides corresponding to the synthetic peptide antigens exist in Colostrinin we carried out tests to determine whether certain of the antibodies produced a reaction in Colostrinin itself.

We studied the rate at which the peptides A-4, B-7, B-8 and B-9 disappeared from colostrum produced in sheep. The colostrum was collected from the mother's milk at 24 hours, 48 hours and 72 hours post parturition, and the level of the peptides was measured. The peptide level was measured by means of an antigen-antibody reaction, using the antibodies produced by the method of Example 3. The result are shown in Table 7.

TABLE 7

| Peptide: | 24 Hour Titre | 48 Hour Titre | 72 Hour Titre |
|---|---|---|---|
| A-4 | 12800 | 6400 | 3200 |
| B-7 | 12800 | 6400 | 3200 |
| B-8 | 12800 | 3200 | 3200 |
| B-9 | 12800 | 12800 | 3200 |

These results demonstrated that antibodies had recognised the amino acid sequences A-4, B-7, B-8 and B-9, and that the concentration of the peptide had diminished over time, owing to binding of the antibody with the peptide.

It will be appreciated that the invention described above may be modified.

What is claimed is:

1. A peptide, in substantially isolated form, which substantially includes the amino-terminal amino acid sequence: LQTPQPLLQVMMEPQGD-OH (SEQ ID 1); MPQNFYKLPQM (SEQ ID 2); VLEMKFPPPPQETVT (SEQ ID 3); LKPFPKLKVEVFPFP (SEQ ID 4); SEQP (SEQ ID 5); DKE (SEQ ID 6); DPPPPQS (SEQ ID 7); LNF (SEQ ID 8); VLPPNVG (SEQ ID 9); KYKLQPE (SEQ ID 10); SEEMP (SEQ ID 11); DSQPPV (SEQ ID 12); FPPPK (SEQ ID 13); VVMEV (SEQ ID 14); DLEMPVLPVEPFPFV (SEQ ID 15); LFFFLPVVNVLP (SEQ ID 16); MQPPPLP (SEQ ID 17); DQPPDVEKPDLQPFQVQS (SEQ ID 18); VYPFTGPIPN (SEQ ID 19); SLPQNILPL (SEQ ID 20); TQTPVVVPPF (SEQ ID 21); LQPEIMGVPKVKETMVPK (SEQ ID 22); HKEMPFPKYPVEPFTESQ (SEQ ID 23); SLTLTDVEKLHLPLPLVQ (SEQ ID 24); SWMHQPP (SEQ ID 25); QPLPPTVMFP (SEQ ID 26); MHQPPQPLPPTVMFP (SEQ ID 27); PQSVLS (SEQ ID 28); LSQPKVLPVPQKAVPQRDMPIQ (SEQ ID 29); AFLLYQE (SEQ ID 30); FLLYQEPVLGPVR (SEQ ID 31); RGPFPILV (SEQ ID 32); ATFNRYQDDHGEEILKSL (SEQ ID 33).

2. A peptide, in substantially isolated form, which substantially includes the amino acid sequence: LQTPQPLLQVMMEPQGD (SEQ ID 1); MPQNFYKLPQM (SEQ ID 2); VLEMKFPPPPQETVT (SEQ ID 3); LKPFPKLKVEVFPFP (SEQ ID 4); DPPPPQS (SEQ ID 7); VLPPNVG (SEQ ID 9); KYKLQPE (SEQ ID 10); DSQPPV (SEQ ID 12); DLEMPVLPVEPFPFV (SEQ ID 15), LFFFLPVVNVLP (SEQ ID 16); MQPPPLP (SEQ ID 17); DQPPDVEKPDLQPFQVQS (SEQ ID 18).

3. A peptide, in substantially isolated form, which substantially entirely consists of the amino acid sequence: LQTPQPLLQVMMEPQGD (SEQ ID 1); MPQNFYKLPQM (SEQ ID 2); VLEMKFPPPPQETVT (SEQ ID 3); LKPFPKLKVEVFPFP (SEQ ID 4); SEQP (SEQ ID 5); DKE (SEQ ID 6); DPPPPQS (SEQ ID 7); LNF (SEQ ID 8); VLPPNVG (SEQ ID 9); KYKLQPE (SEQ ID 10); SEEMP (SEQ ID 11); DSQPPV (SEQ ID 12); FPPPK (SEQ ID 13); VVMEV (SEQ ID 14); DLEMPVLPVEPFPFV (SEQ ID 15); LFFFLPVVNVLP (SEQ ID 16); MQPPPLP (SEQ ID 17); DQPPDVEKPDLQPFQVQS (SEQ ID 18); VYPFTGPIPN (SEQ ID 19); SLPQNILPL (SEQ ID 20); TQTPVVVPPF (SEQ ID 21); LQPEIMGVPKVKETMVPK (SEQ ID 22); HKEMPFPKYPVEPFTESQ (SEQ ID 23); SLTLTDVEKLHLPLPLVQ (SEQ ID 24); SWMHQPP (SEQ ID 25); QPLPPTVMFP (SEQ ID 26); MHQPPQPLPPTVMFP (SEQ ID 27); PQSVLS (SEQ ID 28); LSQPKVLPVPQKAVPQRDMPIQ (SEQ ID 29); AFLLYQE (SEQ ID 30); FLLYQEPVLGPVR (SEQ ID 31); RGPFPILV (SEQ ID 32); ATFNRYQDDHGEEILKSL (SEQ ID 33).

4. A peptide according to claim 1 when obtained by a synthetic process.

5. A peptide obtained by a synthetic process, which substantially includes the amino-terminal amino acid sequence: LQTPQPLLQVMMEPQGD-OH (SEQ ID 1); MPQNFYKLPQM (SEQ ID 2); VLEMKFPPPPQETVT (SEQ ID 3); LKPFPKLKVEVFPFP (SEQ ID 4); SEQP (SEQ ID 5); DKE (SEQ ID 6); DPPPPQS (SEQ ID 7); LNF (SEQ ID 8); VLPPNVG (SEQ ID 9); KYKLQPE (SEQ ID 10); SEEMP (SEQ ID 11); DSQPPV (SEQ ID 12); FPPPK (SEQ ID 13); VVMEV (SEQ ID 14); DLEMPVLPVEPFPFV (SEQ ID 15); LFFFLPVVNVLP (SEQ ID 16); MQPPPLP (SEQ ID 17); DQPPDVEKPDLQPFQVQS (SEQ ID 18); VYPFTGPIPN (SEQ ID 19); SLPQNILPL (SEQ ID 20); TQTPVVVPPF (SEQ ID 21); LQPEIMGVPKVKETMVPK (SEQ ID 22); HKEMPFPKYPVEPFTESQ (SEQ ID 23); SLTLTDVEKLHLPLPLVQ (SEQ ID 24); SWMHQPP (SEQ ID 25); QPLPPTVMFP (SEQ ID 26); MHQPPQPLPPTVMFP (SEQ ID 27); PQSVLS (SEQ ID 28); LSQPKVLPVPQKAVPQRDMPIQ (SEQ ID 29); AFLLYQE (SEQ ID 30); FLLYQEPVLGPVR (SEQ ID 31); RGPFPILV (SEQ ID 32); ATFNRYQDDHGEEILKSL (SEQ ID 33).

6. A peptide obtained by a synthetic process, which substantially includes the amino acid sequence: LQTPQPLLQVMMEPQGD-OH (SEQ ID 1); MPQNFYKLPQM (SEQ ID 2); VLEMKFPPPPQETVT (SEQ ID 3); LKPFPKLKVEVFPFP (SEQ ID 4); DPPPPQS (SEQ ID 7); VLPPNVG (SEQ ID 9); KYKLQPE (SEQ ID 10); DSQPPV (SEQ ID 12); DLEMPVLPVEPFPFV (SEQ ID 15); LFFFLPVVNVLP (SEQ ID 16); MQPPPLP (SEQ ID 17); DQPPDVEKPDLQPFQVQS (SEQ ID 18).

7. A peptide obtained by a synthetic process, which substantially entirely consists of the amino acid sequence: LQTPQPLLQVMMEPQGD-OH (SEQ ID 1); MPQNFYKLPQM (SEQ ID 2); VLEMKFPPPPQETVT (SEQ ID 3); LKPFPKLKVEVFPFP (SEQ ID 4); SEQP (SEQ ID 5); DKE (SEQ ID 6); DPPPPQS (SEQ ID 7); LNF (SEQ ID 8); VLPPNVG (SEQ ID 9); KYKLQPE (SEQ ID 10); SEEMP (SEQ ID 11); DSQPPV (SEQ ID 12); FPPPK (SEQ ID 13); VVMEV (SEQ ID 14); DLEMPVLPVEPFPFV (SEQ ID 15); LFFFLPVVNVLP (SEQ ID 16); MQPPPLP (SEQ ID 17); DQPPDVEKPDLQPFQVQS (SEQ ID 18); VYPFTGPIPN (SEQ ID 19); SLPQNILPL (SEQ ID 20); TQTPVVVPPF (SEQ ID 21); LQPEIMGVPKVKETMVPK (SEQ ID 22); HKEMPFPKYPVEPFTESQ (SEQ ID 23); SLTLTDVEKLHLPLPLVQ (SEQ ID 24); SWMHQPP (SEQ ID 25); QPLPPTVMFP (SEQ ID 26); MHQPPQPLPPTVMFP (SEQ ID 27); PQSVLS (SEQ ID 28); LSQPKVLPVPQKAVPQRDMPIQ (SEQ ID 29); AFLLYQE (SEQ ID 30); FLLYQEPVLGPVR (SEQ ID 31); RGPFPILV (SEQ ID 32); ATFNRYQDDHGEEILKSL (SEQ ID 33).

8. A peptide comprising: NH₂-(Ac)CLQTPQPLLQVMMEPQGD-OH (SEQ ID 34); NH₂-(Ac)CMPQNFYKLPQM-OH (SEQ. ID 35); NH₂-(Ac)CVLEMKFPPPPQETVT-OH (SEQ ID 36); NH₂-(Ac)CLKPFPKLKVEVFPFP-OH (SEQ ID 37); NH₂-SEQPGGGC-OH (SEQ ID 38); NH₂-(Ac)CGVLPPNVG-OH (SEQ ID 39); NH2-(Ac)CGGGKYKLQE-OH (SEQ ID 40); NH₂-(Ac)CGGGSEEMP(amide)-OH (SEQ ID 41); NH₂-(Ac)CGGGDSQPPV-OH (SEQ ID 42); NH₂-CFPPPKGGGC-OH (SEQ ID 43); NH₂-(Ac)CGGGWMEV-OH (SEQ ID 44); NH₂-(Ac)CDLEMPVLPVEPFPFV-OH (SEQ ID 45); NH₂-(Ac)CLFFFLPWNVLPI-OH (SEQ ID 46); NH₂-(Ac)CMQPPPLP-OH (SEQ ID 47); NH₂-(Ac)CDQPPDVEKPDLQPFQVQS-OH (SEQ ID 48); NH₂-(Ac)CGAFLLYQE-OH (SEQ ID 49); NH₂-(Ac)CATFNRYQDDHGEEILKSL-OH (SEQ ID 50).

9. A peptide according to claim 1, for use as a medicament.

10. A peptide according to claim 9, for use in the treatment of chronic disorders of the central nervous system.

11. A peptide according to claim 10, for use in the treatment of neurological disorders and/or mental disorders.

12. A peptide according to claim 9, for use in the treatment of dementia and/or neurodegenerative diseases.

13. A peptide according to claim 9, for use in the treatment of Alzheimer's disease and/or motor neurone disease.

14. A peptide according to claim 9, for use in the treatment of psychosis and/or neurosis.

15. A peptide according to claim 9, for use in the treatment of chronic disorders of the immune system.

16. A peptide according to claim 9, for use in the treatment of diseases with a bacterial and viral aetiology, and/or for use in the treatment of acquired immunological deficiencies.

17. A peptide according to claim 9, for use in the treatment of chronic bacterial and/or viral infections.

18. A peptide according to claim 9, for use in the treatment of diseases characterised by the presence of P-amyloid plaque.

19. The use of a peptide according to claim 1 in the manufacture of a medicament for the treatment of chronic disorders of the central nervous system.

20. The use of a peptide according to claim 1 in the manufacture of a medicament for the treatment of chronic disorders of the immune system.

21. A method of treating disorders of the central nervous system and/or of the immune system, comprising administering a therapeutically effective amount of a peptide according to claim 1 to a patient.

22. A composition comprising a peptide according to claim 1 in combination with a physiologically acceptable carrier.

23. A composition comprising two or more peptides according to claim 1 in combination with a physiologically acceptable carrier.

24. A composition according to claim 22 in a form suitable for injection.

25. A composition according to claim 22 in a form suitable for absorption through the mucosa of the oral/nasopharyngeal cavity and/or in a form suitable for absorption in the alimentary canal.

26. A composition according to claim 22 in the form of a tablet, lozenge, gel, patch or plaster.

27. A composition according to claim 22 in a form suitable for topical application.

28. The use of a peptide according to claim 1 as a dietary supplement.

29. The use of a peptide according to claim 1 as a dietary supplement for babies, small children, adults who have been subjected to chemotherapy and/or adults who have suffered from cahexia, or weight loss due to chronic disease.

30. A dietary supplement comprising an orally ingestible combination of a peptide according to claim 1 combination with a physiologically acceptable carrier.

31. An antibody which binds to a peptide according to claim 1.

32. An antibody obtainable by using a peptide according to claim 1 as an antigen.

33. A peptide containing the amino acid sequence LQTPQPLLQVMMEPQGD; DPPPPOS; and/or LFFFLPWNVLP for use as an immunosuppressant.

34. A peptide containing the amino acid sequence LQTPQPLLQVMMEPQGD; DPPPPQS; and/or LFFFLPVVNVLP for use in the treatment of autoimmune disorder.

35. A peptide containing the amino acid sequence LQTPQPLLQVMMEPQGD; DPPPPQS; and/or LFFFLPVVNVLP for use in suppressing the rejection of transplanting organs.
